(19) United States
(12) Patent Application Publication
(10) Pub. No.: US 2024/0374645 A1
(43) Pub. Date: Nov. 14, 2024

(54) BIOLOGICALLY RELEVANT ORTHOGONAL CYTOKINE/RECEPTOR PAIRS

(71) Applicant: The Board of Trustees of the Leland Stanford Junior University, Stanford, CA (US)

(72) Inventors: Kenan Christopher Garcia, Menlo Park, CA (US); Jonathan Sockolosky, San Francisco, CA (US); Lora Picton, Foster City, CA (US)

(21) Appl. No.: 18/789,470

(22) Filed: Jul. 30, 2024

Related U.S. Application Data
(63) Continuation of application No. 16/977,385, filed on Sep. 1, 2020, now abandoned.
Application No. 16/977,385 is a division of application No. 15/916,689, filed on Mar. 9, 2018, now Pat. No. 10,869,887.

(51) Int. Cl. A61K 35/17; A61K 38/17; A61K 38/20; A61K 39/00; C07K 14/55; C12N 5/0783; C12N 15/10

(52) U.S. Cl. CPC A61K 35/17; A61K 38/1793; A61K 38/2013; C07K 14/55; C12N 5/0638; C12N 15/1034; A61K 2039/5156; A61K 2300/00; C12N 2740/10043

(57) ABSTRACT

Engineered orthogonal cytokine receptor/ligand pairs, and methods of use thereof, are provided.

### CROSS-REFERENCE

This application is a Continuation and claims the benefit of application Ser. No. 16/977,385, filed Sep. 1, 2020, which claims the benefit of PCT Application No. PCT/US2019/021451, filed Mar. 8, 2019, which is a Division of application Ser. No. 15/916,689, filed Mar. 9, 2018, now U.S. Pat. No. 10,869,887, issued Dec. 22, 2020, which applications are incorporated herein by reference in their entirety.

### FEDERALLY SPONSORED RESEARCH AND DEVELOPMENT

This invention was made with Government support under contract AI513210 awarded by the National Institutes of Health. The Government has certain rights in the invention.

### INCORPORATION BY REFERENCE OF SEQUENCE LISTING

A Sequence Listing is provided herewith as a Sequence Listing XML, STAN-1246US2CON2_SEQ_LIST created on Jul. 29, 2024, and having a size of 26,597 bytes. The contents of the Sequence Listing XML are incorporated herein by reference in their entirety.

### BACKGROUND

The manipulation of cells, particularly immune cells, to differentiate, develop specialized functions and expand in numbers is of great clinical interest. Many protein factors that affect these activities are known in the art, including in particular cytokines and chemokines. However, these signaling molecules also have pleiotropic effects on cells not targeted for manipulation, and thus methods of selectively activating signaling in a targeted cell population are desirable. In particular, engineering of T cells to carry out controlled behaviors is of interest. For example, in adoptive immunotherapy T cells are isolated from blood, processed ex vivo, and re-infused into patients. T cells have been engineered for use in therapeutic applications such as the recognition and killing of cancer cells, intracellular pathogens and cells involved in auto-immunity.

A critical challenge in cell based therapies is engineering into adoptively transferred cells a desired behavior, such as activation, expansion, etc., that is protected from endogenous signaling pathways, that does not affect non-targeted endogenous cells, and that can be controlled once administered to a patient. This is particularly relevant for T cell engineering because of developmental plasticity and the immense impact that environmental factors play in determining T cell fate, function, and localization.

The ability to manipulate proteins to bind and respond to modified ligands in a manner independent, or orthogonal, from the influence of the native proteins or ligands, constitutes a significant challenge in protein engineering. To date, numerous synthetic ligand-ortholog receptor pairs have been created that are orthogonal to the analogous natural interaction. Among the proteins used for this work, are included nuclear hormone receptors and G-protein coupled receptors. Despite the extensive work carried out to engineer receptors that are activated by synthetic small molecule ligands, the engineering of pairs of biologically relevant proteins remains a significant challenge.

### SUMMARY

Engineered orthogonal cytokine receptor/ligand pairs, and methods of use thereof, are provided. An engineered (orthogonal) cytokine specifically binds to a counterpart engineered (orthogonal) receptor. Upon binding, the orthogonal receptor activates signaling that is transduced through native cellular elements to provide for a biological activity that mimics that native response, but which is specific to an engineered cell expressing the orthogonal receptor. The orthogonal receptor exhibits significantly reduced binding to the endogenous counterpart cytokine, including the native counterpart of the orthogonal cytokine, while the orthogonal cytokine exhibits significantly reduced binding to any endogenous receptors, including the native counterpart of the orthogonal receptor. In some embodiments, the affinity of the orthogonal cytokine for the orthogonal receptor is comparable to the affinity of the native cytokine for the native receptor.

The process for engineering an orthogonal cytokine receptor pair may comprise the steps of: (a) engineering amino acid changes into a native receptor to disrupt binding to the native cytokine; (b) generating multiple cytokine analogs that possess selective amino acid changes into the native cytokine at contact residues for receptor binding, (c) selecting for cytokine orthologs that bind to the ortholog receptor; (d) discarding ortholog cytokines that bind significantly to the native receptor, or alternatively to steps (c) and (d); (e) selecting for receptor orthologs that bind the ortholog cytokine; (f) discarding ortholog receptors that bind to the native cytokine. In preferred embodiments, knowledge of the structure of the cytokine/receptor complex is used to select amino acid positions for site-directed or error prone mutagenesis. Conveniently a yeast display system can be used for the selection process, although other display and selection methods are also useful.

In some embodiments, an engineered cell is provided, in which the cell has been modified by introduction of an orthogonal receptor of the invention. Any cell can be used for this purpose. In some embodiments the cell is a T cell, including without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, natural T_{Reg}, inducible T_{Reg}; memory T cells, e.g. central memory T cells, effector memory T cells, NKT cells, γδ T cells and engineered variants of such T-cells including CAR-T cells; etc. In other embodiments the engineered cell is a stem cell, e.g. a hematopoietic stem cell, an NK cell, a macrophage, or a dendritic cell. In some embodiments the cell is genetically modified in an ex vivo procedure, prior to transfer into a subject. The engineered cell can be provided in a unit dose for therapy, and can be allogeneic, autologous, etc. with respect to an intended recipient.

In some embodiments a vector comprising a polynucleotide coding sequence that encodes the orthogonal receptor is provided, where the coding sequence is operably linked to a promoter active in the desired cell. Various vectors are known in the art and can be used for this purpose, e.g. viral vectors, plasmid vectors, minicircle vectors, which vectors can be integrated into the target cell genome, or can be episomally maintained. The receptor encoding vector may be provided in a kit, combined with a vector encoding an orthogonal cytokine that binds to and activates the receptor. In some embodiments the coding sequence for the orthogonal cytokine is operably linked to a high expression promoter and may be optimized for production. In other embodiments, a kit is provided in which the vector encoding the orthogonal receptor is provided with a purified composition of the orthogonal cytokine, e.g. in a unit dose, packaged for administration to a patient (e.g. a prefilled syringe). In still some other embodiments, a kit is provided in which the vector encoding the orthogonal receptor is provided with a vector encoding the orthogonal cytokine to enable expression of the orthogonal receptor in a cell and also expression of the orthogonal cytokine intended for secretion by the same cell to enable autocrine orthogonal cytokine-receptor signaling.

In some embodiments a therapeutic method is provided, the method comprising introducing into a recipient in need thereof of an engineered cell population, wherein the cell population has been modified by introduction of a sequence encoding an orthogonal receptor of the invention. The cell population may be engineered ex vivo, and is usually autologous or allogeneic with respect to the recipient. In some embodiments, the introduced cell population is contacted with the cognate orthogonal cytokine in vivo, following administration of the engineered cells. An advantage of the present invention is a lack of cross-reactivity between the orthogonal cytokine and the native receptor.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1-1 (ii). Orthogonal IL-2/IL-2 receptor pairs to control T cell expansion.

FIG. 2. Work flow for engineering orthogonal IL-2/IL-2Rβ pairs.

FIG. 3. Sequences of orthogonal mouse IL-2Rβ variants.

FIG. 4. mIL-2Rβ H134D Y135F mutations abrogate wt mIL-2 binding.

FIG. 5. Work flow for engineering orthogonal IL-2/IL-2Rβ pairs.

FIG. 6. Sequences of characterized orthogonal mouse IL-2 variants.

FIG. 7. OrthoIL-2 variants bind orthoIL-2Rβ with affinity similar to or greater than the wild-type IL-2 and IL-2Rβ interaction.

FIG. 8. orthoIL-2 variants exhibit blunted activity (phosphoSTAT5) on wild-type CD25 positive and CD25 negative Splenocytes

FIG. 9. Generation of orthoIL-2R expressing mouse CTLL-2 T cells.

FIG. 10. First set of orthoIL-2 variants are selective for ortho T cells.

FIG. 11. OrthoIL-2 variants induce selective STAT5 phosphorylation on orthoIL-2Rβ expressing CTLL-2 cells

FIG. 12. Primary lymph node derived T cells engineered to express orthoIL-2Rβ (H134D Y135F).

FIG. 13. OrthoIL-2 variants induce selective STAT5 phosphorylation on orthoIL-2Rβ expressing primary mouse T cells.

FIG. 14. orthoIL-2 variants induce selective cell growth of orthoIL-2Rβ expressing CTLL-2 cells compared to wild-type T cells.

FIG. 15. Alignment of mouse and human reference IL-2Rβ/IL-2 sequences. A partial sequence of human IL-2Rβ is provided as SEQ ID NO:1, residues 1-235; a partial sequence of mouse IL-2Rβ is provided as SEQ ID NO:2, residues 1-238. Mouse IL-2 is provided as SEQ ID NO: 3. Human IL-2 is provided as SEQ ID NO:4.

FIG. 16A-16D. Yeast evolution of orthogonal human IL-2 pairs. (FIG. 16A) FACS analysis of yeast-displayed wild-type human IL-2 binding to wild-type (blue histogram) but not the ortho (red histogram) human IL-2Rβ H133D Y134F mutant tetramers. (FIG. 16B) Libraries of human IL-2 mutants (˜1⁸ mutants) that randomize IL-2 residues predicted to be in proximity to or contacting the human IL-2Rβ HY mutant were displayed on the surface of yeast. After successive rounds of rounds of both positive (against ortho hIL-2Rβ) and negative (against wild-type hIL-2Rβ) selection, we obtained yeast-displayed human IL-2 mutants that bind ortho (red histogram) but not wild-type (blue histogram) human IL-2Rβ tetramers. (FIG. 16C, 16D) ortho hIL-2 mutants were subsequently isolated and sequenced from the yeast library. A consensus set of mutations were identified indicating a convergence of ortho hIL-2 sequences capable of binding the ortho hIL-2Rβ.

FIG. 17. In vivo mouse model used to demonstrate selective expansion or increased survival of orthogonal IL-2Rb expressing T cells in mice. Donor cells were isolated from the spleen of wild-type C57BL/6J mice that express CD45.2, activated ex vivo with CD3/CD28, transduced with retrovirus encoding orthogonal IL-2Rb-IRES-YFP, expanded for 2 days in 100 IU/mL mIL-2, and purified using a mouse CD8 T cell isolation kit (Miltenyi). An ˜ 1:1 mixture of wild-type (CD45.2 positive, YFP negative) and orthogonal IL-2Rb expressing T cells (CD45.2 positive, YFP positive) were adoptively transferred into recipient BL6.Rag2^{-/-} x IL2rg^{-/-} CD45.1 mice via retro-orbital injection. PBS, wild-type mIL-2 (150,000 IU/mouse), or orthoIL-2 clone 1G12/149 (1,000,000 IU/mouse), were injected IP daily beginning immediately after T cell transfer (d0) and at 24 hr intervals for 5 consecutive days (up to d4). Mice were sacrificed on d5 and d7 and total donor T cell counts in the blood and spleen of mice were quantified by flow cytometry.

FIG. 18A-18B. Gating strategy used to quantify donor T cell expansion in recipient mice. Single cell suspension from mouse blood and spleen were prepared and stained with CD45.2-pacific blue for 1 hr at 4 C for identification of donor T cells. Immediately prior to flow cytometry cells were incubated with a 1:2000 dilution of propidium iodide (PI) for live/dead exclusion. Cells were gated based on forward and side scatter (SSC-A v FSC-A), singlets (FSC-A v FSC-H), live cells (PI negative), and the total number of wild-type T cells (CD45.2 positive, YFP-negative) and orthogonal T cells (CD45.2 positive, YFP-positive) was quantified via FACS. **p<0.01, ***p<0.001, ****p<0.0001, determined by one-way ANOVA using Prism.

FIG. 19. orthoIL-2 clone 1G12/149 selectively expands orthogonal but not wild-type T cells in mice. The number of wild-type and orthogonal T cells in blood (10³ cells/μL) and spleen (total number of cells per spleen) were quantified via flow cytometry as described in FIG. 18. The ratio of orthogonal T cells to wild-type T cells was determined by dividing the total number orthogonal T cells by the total number of wild-type T cells in the blood and spleen. A ratio greater than 1 indicates selective expansion of orthogonal T cells, which is achieved with orthoIL-2 clone 1G12/149. Total number of viable cells in the blood (left) and spleen (right) at day 5 (top) and day 7 (bottom) were quantified via flow cytometry. Treatment with wild-type IL-2 results in expansion of both wild-type and ortho T cells compared to a PBS control, whereas treatment with orthoIL-2 clone 1G12/149 selectively expands ortho T cells with limited activity on wild-type T cells.

FIG. 20A-20B. Orthogonal IL-2 has selective activity on orthogonal IL-2Rβ T cells. (FIG. 20A) FACS analysis of primary, spleen derived mouse T cells isolated from IL-2 KO NOD mice and virally transduced to express orthoIL-2Rβ, which can be confirmed using an IRES-YFP reporter and surface staining for IL-2Rβ. The T cells also retain expression of wild-type IL-2Rβ (FIG. 20B) orthoIL-2 induces selective STAT5 phosphorylation on orthoIL-2Rβ expressing T cells with blunted to no activity on wild-type T cells.

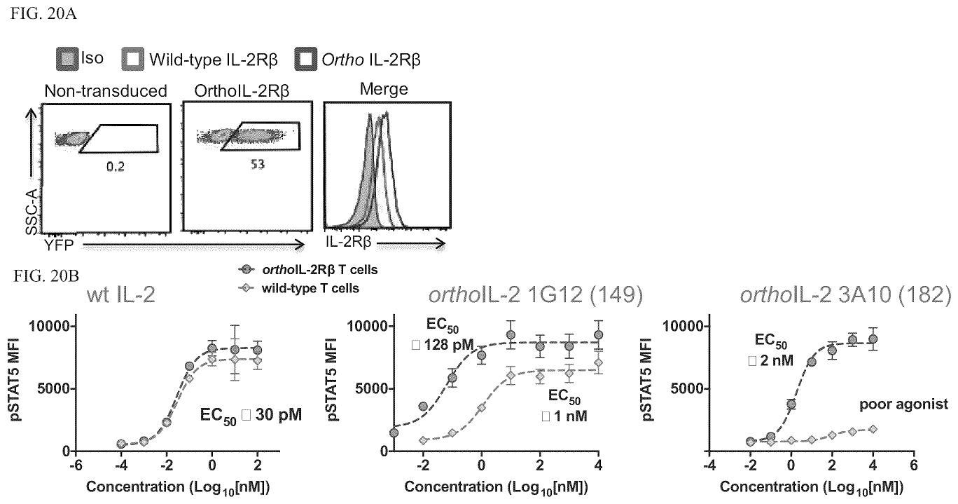

FIG. 21A-21B. Orthogonal IL-2 selectively expands orthogonal IL-2Rβ T cells in vitro. (FIG. 21A) FACS analysis of primary, spleen derived mouse T cells virally transduced to express orthoIL-2Rβ, which can be confirmed using an IRES-YFP. The mixture of transduced and un-transduced T cells were cultured for 5 days in various concentrations of wild-type, orthoIL-2 clone 1G12, or 3A10 and analyzed by FACS. IL-2 expands both wild-type and ortho T cells, whereas only ortho T cells expand when cultured in orthoIL-2 3A10 whereas orthoIL-2 1G12 selectively expands ortho T cells with significantly reduced activity on wild-type T cells. The FACS plot show correspond to culture in 100 nM IL-2, 64 pM orthoIL-2 1G12, and 10 μM orthoIL-2 3A10. (FIG. 21B) Wild-type and ortho T cell proliferation dose-response to wild-type and orthoIL-2 clones 1G12 and 3A10 after 5 days of culture in increasing concentrations of cytokine. IL-2 expands both wild-type and ortho T cells with equal potency, orthoIL-2 1G12 selectively expands ortho T cells, and orthoIL-2 3A10 specifically expands ortho T cells.

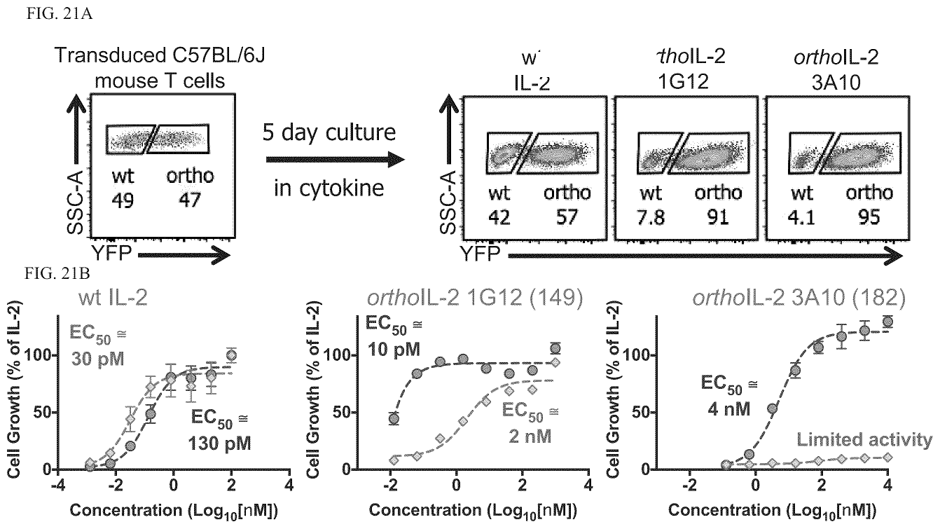

FIG. 22A-22E. Ortho human IL-2 signals through the orthoIL-2R expressed in YT cells in vitro. Dose-response of STAT5 phosphorylation after 20 min of stimulation. The phosphorylation of Stat5 was measured in the YT human NK cell line, expressing human CD25 (YT+), without (YFP−, WT) or with (YFP+, ortho) human ortho IL-2Rb. (FIG. 22A) Mouse serum albumin (MSA) fusions of human IL-2, or orthogonal variants (FIG. 22B) 1A1, (FIG. 22C) 1C7, (FIG. 22D) SQVLKA or (FIG. 22E) SQVKqA were titrated in RPMI complete media and added to the cells. The mean fluorescence intensities (MFI) of APC-pStat5 staining for WT (YFP−) and ortho Rb (YFP+) cells were plotted versus the concentration of cytokine, and fit to a log (agonist) vs. response (three parameters) model using Prism 5 (GraphPad). 1C7 was run on a separate day from the other proteins, and was normalized to wild type IL-2 staining run on both days. All data are presented as mean (n=3)+SD.

FIG. 23A-23B. Ortho human IL-2 preferentially expands human PBMCs expressing the ortho IL-2R. Human PBMCs were isolated, activated and transformed with retrovirus containing ortho human IL-2Rβ with an IRES YFP (YFP+). Initial ratio of YFP+ cells to total live cells was 20%. 5×10⁵ cells were plated with the indicated concentrations of MSA-human IL-2 (circles) or ortho variants MSA-SQVLKA (diamonds), MSA-SQVLqA (squares) or MSA-1A1 (triangles) on day 1, and re-fed the same concentration on day 3. On day 5 the plate was read by flow cytometry. (FIG. 23A) The ratio of YFP+ (ortho expressing) cells to total live cells was calculated, and the mean (n=4)±SD was plotted versus the concentration (left). (FIG. 23B) Total live cell counts (mean (n=4)±SD) were also plotted versus the cytokine concentration (right). The orthogonal cytokines were unable to support as much total cell growth as wild type MSA-hIL-2 at the same concentration.

### DETAILED DESCRIPTION OF THE EMBODIMENTS

In order for the present disclosure to be more readily understood, certain terms and phrases are defined below as well as throughout the specification. The definitions provided herein are non-limiting and should be read in view of what one of skill in the art would know at the time of invention.

### Definitions

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Cytokine receptor and ligand pairs include, without limitation, the following receptors:

| Cytokine | Receptor subunits |
|---|---|
| IL-1-like | CD121a, CDw121b |
| IL-1α | CD121a, CDw121b |
| IL-1β | CD121a, CDw121b |
| IL-1RA | CD121a |
| IL-18 | IL-18Rα, β |
| IL-2 | CD25, 122, 132 |
| IL-4 | CD124, 213a13, 132 |
| IL-7 | CD127, 132 |
| IL-9 | IL-9R, CD132 |
| IL-13 | CD213a1, 213a2, |
| | CD1243, 132 |
| IL-15 | IL-15Ra, CD122, 132 |
| IL-3 | CD123, CDw131 |
| IL-5 | CDw125, 131 |
| GM-CSF | CD116, CDw131 |
| IL-6 | CD126, 130 |
| IL-11 | IL-11Ra, CD130 |
| G-CSF | CD114 |
| IL-12 | CD212 |
| LIF | LIFR, CD130 |
| OSM | OSMR, CD130 |
| IL-10 | CDw210 |
| IL-20 | IL-20Rα, β |
| IL-14 | IL-14R |
| IL-16 | CD4 |
| IL-17 | CDw217 |
| IFN-α | CD118 |
| IFN-β | CD118 |
| IFN-γ | CDw119 |
| CD154 | CD40 |
| LT-β | LTβR |
| TNF-α | CD120a, b |
| TNF-β | CD120a, b |
| 4-1BBL | CDw137 (4-1BB) |
| APRIL | BCMA, TACI |
| CD70 | CD27 |
| CD153 | CD30 |
| CD178 | CD95 (Fas) |
| GITRL | GITR |
| LIGHT | LTbR, HVEM |
| OX40L | 0X40 |
| TALL-1 | BCMA, TACI |
| TRAIL | TRAILR1-4 |
| TWEAK | Apo3 |
| TRANCE | RANK, OPG |
| TGF-β1 | TGF-βR1 |
| TGF-β2 | TGF-βR2 |
| TGF-β3 | TGF-βR3 |
| Epo | EpoR |
| Tpo | TpoR |
| Flt-3L | Flt-3 |
| SCF | CD117 |
| M-CSF | CD115 |
| MSP | CDw136 |

An “ortholog”, or “orthogonal cytokine/receptor pair” refers to a genetically engineered pair of proteins that are modified by amino acid changes to (a) exhibit significantly reduced affinity to the native cytokine or cognate receptor; and (b) to specifically bind to the counterpart engineered (orthogonal) ligand or receptor. Upon binding of the orthogonal ligand, the orthogonal receptor activates signaling that is transduced through native cellular elements to provide for a biological activity that mimics that native response, but which is specific to an engineered cell expressing the orthogonal receptor.

An orthogonal receptor exhibits significantly reduced binding to its cognate native cytokine ligand, while an orthogonal cytokine exhibits significantly reduced binding to its cognate native receptor(s). In some embodiments, the affinity of the orthogonal cytokine for the cognate orthogonal receptor is comparable to the affinity of the native cytokine for the native receptor, e.g. having an affinity that is least about 1% of the native cytokine receptor pair affinity, at least about 5%, at least about 10%, at least about 25%, at least about 50%, at least about 75%, at least about 100%, and may be higher, e.g. 2×, 3×, 4×, 5×, 10× or more of the affinity of the native cytokine for the native receptor

As used herein, “do not bind” or “incapable of binding” refers to no detectable binding, or an insignificant binding, i.e., having a binding affinity much lower than that of the natural ligand. The affinity can be determined with competitive binding experiments that measure the binding of a receptor with a single concentration of labeled ligand in the presence of various concentrations of unlabeled ligand. Typically, the concentration of unlabeled ligand varies over at least six orders of magnitude. Through competitive binding experiments, IC₅₀ can be determined. As used herein, “IC₅₀” refers to the concentration of the unlabeled ligand that is required for 50% inhibition of the association between receptor and the labeled ligand. IC₅₀ is an indicator of the ligand-receptor binding affinity. Low IC₅₀ represents high affinity, while high IC₅₀ represents low affinity.

As used herein the term “specifically binds” refers to the degree of selectivity or affinity for which one molecule binds to another. In the context of binding pairs (e.g. a ligand/receptor, antibody/antigen, antibody/ligand, antibody/receptor binding pairs) a first molecule of a binding pair is said to specifically bind to a second molecule of a binding pair when the first molecule of the binding pair does not bind in a significant amount to other components present in the sample.

A first molecule of a binding pair is said to specifically bind to a second molecule of a binding pair when the first molecule of the binding pair when the affinity of the first molecule for the second molecule is at least two-fold greater, at least ten times greater, at least 20-times greater, or at least 100-times greater than the affinity of the first molecule for other components present in the sample. In a particular embodiment, where the first molecule of the binding pair is an antibody, the antibody specifically binds to the second molecule of the binding pair (e.g. a protein, antigen, ligand, or receptor) if the affinity of the antibody for the second molecule of the binding pair is greater than about 10⁹ liters/mole, alternatively greater than about 10¹⁰ liters/mole, greater than about 10¹¹ liters/mole, greater than about 10¹² liters/mole as determined by, e.g., Scatchard analysis (Munsen, et al. 1980 Analyt. Biochem. 107:220-239). Specific binding may be assessed using techniques known in the art including but not limited to competition ELISA, BIACORE® assays and/or KINEXA® assays.

As used herein, the term “exhibits significantly reduced binding” is used with respect to the affinity of the binding of the orthogonal ligand to the orthogonal receptor relative to the binding of the orthogonal ligand for the naturally occurring form of its cognate receptor. In the practice of the present invention, the term exhibits significantly reduced binding is used to describe the comparative binding and activity of the orthogonal ligand relative to the naturally occurring ligand with respect to the naturally occurring receptor. An orthogonal ligand exhibits significantly reduced binding with respect to the native form of the ligand if the orthogonal ligand binds to the native form of the receptor with less than 20%, alternatively less than about 10%, alternatively less than about 8%, alternatively less than about 6%, alternatively less than about 4%, alternatively less than about 2%, alternatively less than about 1%, alternatively less than about 0.5% of the naturally occurring ligand. Similarly and orthogonal receptor exhibits significantly reduced binding with respect to the native form of the ligand if the native form of the ligand binds to the orthogonal form of the receptor with less than 20%, alternatively less than about 10%, alternatively less than about 8%, alternatively less than about 6%, alternatively less than about 4%, alternatively less than about 2%, alternatively less than about 1%, alternatively less than about 0.5% of the naturally occurring receptor.

An orthogonal IL-2 polypeptide exhibits significantly reduced activation through the native IL-2Rβ. Activity may be measured, for example, in a cell proliferation assay using CTLL-2 mouse cytotoxic T cells, see Gearing, A. J. H. and C. B. Bird (1987) in Lymphokines and Interferons. A Practical Approach. Clemens, M. J. et al. (eds): IRL Press. 295. The specific activity of Recombinant Human IL-2 is approximately 2.1×10⁴ IU/μg, which is calibrated against recombinant human IL-2 WHO International Standard (NIBSC code: 86/500). An orthogonal human IL-2 may have less than 20%, alternatively less than about 10%, alternatively less than about 8%, alternatively less than about 6%, alternatively less than about 4%, alternatively less than about 2%, alternatively less than about 1%, alternatively less than about 0.5% of the activity of WHO International Standard (NIBSC code: 86/500) human IL-2 polypeptide in a comparable assay.

The term “identity,” as used herein in reference to polypeptide or DNA sequences, refers to the sequence identity between two molecules. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “polypeptide,” “protein” or “peptide” refer to any chain of amino acid residues, regardless of its length or post-translational modification (e.g., glycosylation or phosphorylation).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide or may be a modified version of a WT polypeptide. The term variant polypeptide may refer to the polypeptide itself, a composition comprising the polypeptide, or the nucleic acid sequence that encodes it. Preferably, the variant polypeptide comprises at least one amino acid modification compared to the parent polypeptide, e.g. from about one to about ten amino acid modifications, and preferably from about one to about five amino acid modifications compared to the parent. A variant may be at least about 99% identical to the wild-type protein, at least about 98% identical, at least about 97% identical, at least about 95% identical, at least about 90% identical.

By “parent polypeptide”, “parent protein”, “precursor polypeptide”, or “precursor protein” as used herein is meant an unmodified polypeptide that is subsequently modified to generate a variant polypeptide. A parent polypeptide may be a wild-type (or native) polypeptide. Parent polypeptide may refer to the polypeptide itself, compositions that comprise the parent polypeptide, or the amino acid sequence that encodes it.

By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been modified by the hand of man.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

As used herein, a “therapeutically effective amount” refers to that amount of the therapeutic agent, e.g. adoptive T cells or orthogonal cytokines, sufficient to prevent, treat or manage a disease or disorder. A therapeutically effective amount may refer to the amount of therapeutic agent sufficient to delay or minimize the onset of disease, e.g., delay or minimize the spread of cancer, or the amount effect to decrease or increase signaling from a receptor of interest. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the terms “prevent”, “preventing” and “prevention” refer to the prevention of the recurrence or onset of one or more symptoms of a disorder in a subject as result of the administration of a prophylactic or therapeutic agent.

As used herein, the term “in combination” refers to the use of more than one prophylactic and/or therapeutic agents. The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

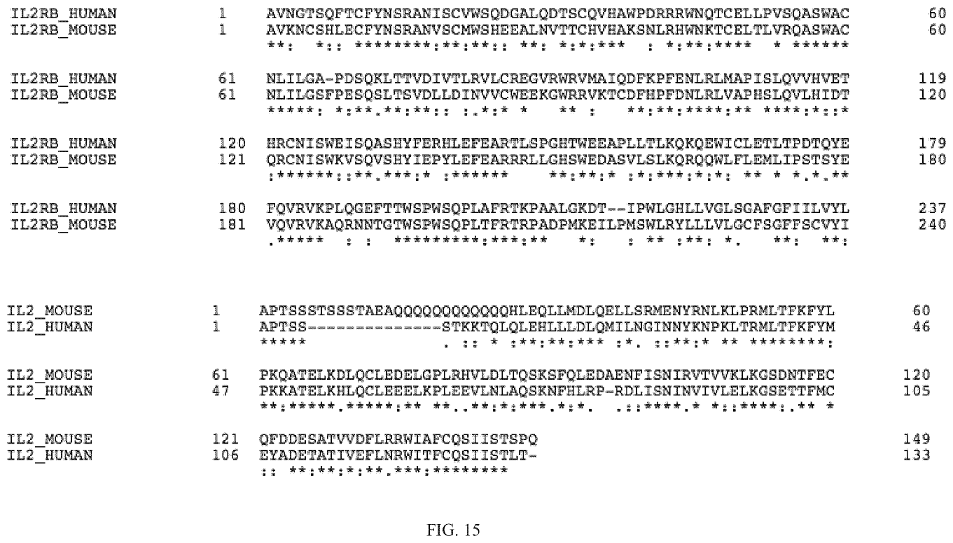

Interleukin 2 (IL-2) is a pluripotent cytokine produced primarily by activated CD4+ T cells and plays a crucial role in producing a normal immune response. IL-2 promotes proliferation and expansion of activated T lymphocytes, potentiates B cell growth, and activates monocytes and natural killer cells. It was by virtue of these activities that IL-2 was tested and is used as an approved treatment of cancer (aldesleukin, Proleukin®). Human IL-2 is synthesized as a precursor polypeptide of 153 amino acids, from which 20 amino acids are removed to generate mature secreted IL-2. As used herein, “IL-2” refers to the native, or wild-type IL-2. Mature human IL-2 occurs as a 133 amino acid sequence (less the signal peptide, consisting of an additional 20 N-terminal amino acids), as described in Fujita, et. al, PNAS USA, 80, 7437-7441 (1983). The amino acid sequence of human IL-2 is found in Genbank under accession locator NP_000577.2. Reference sequences of the human IL-2 (SEQ ID NO:4) and mouse IL-2 (SEQ ID NO:3), human IL-2Rβ (SEQ ID NO:1) and mouse IL-2Rβ (SEQ ID NO:2) are provided in FIG. 15.

IL-2 supports the survival and differentiation of T lymphocytes by initiating cell signaling pathways upon interaction with the IL-2 receptor (IL-2R). IL-2 is used clinically to treat a number of human diseases including cancer and autoimmunity and as an adjuvant to adoptive T cell therapies to promote the survival of transplanted T cells. However, IL-2 can also have apposing effects by activating off-target cell types.

To direct the activity of IL-2 towards a specific T cell subset, the present invention provides engineered orthogonal IL-2 and IL-2 receptor pairs. Orthogonal IL-2 when bound to the orthogonal IL-2 receptor expressed on a cell recapitulates the activity of wild-type IL-2 by inducing potent STAT5 phosphorylation and in vitro proliferation of T cells engineered to express the orthogonal IL-2Rbeta. Orthogonal IL-2 has significantly reduced binding with respect to on ex vivo cultured wild-type CD25 positive or negative mouse T cells, respectively. The studies of the present disclosure indicate that remodeling cytokine receptor interfaces to create interactions that are not present in nature is a viable strategy to direct the activity of a promiscuous cytokine to a T cell subset of interest, thereby enabling precise control over T cell function though genetic engineering.

In addition to IL-2, IL-15 and IL-7 also regulate lymphoid homeostasis and have also been used as adjuvants to potentiate adoptive T cell therapy. IL-2 and IL-15 share the same IL-2R-beta chain. Orthogonal IL-15 can be selected against the identical orthogonal IL-2R-beta used to orthogonalize IL-2. IL-7 utilizes a distinct IL-7R-alpha chain that is a target for orthogonalization.

In some embodiments, the orthogonal cytokine, e.g orthogonal IL2, can be conjugated to additional molecules to provide desired pharmacological properties such as extended half-life. In one embodiment, an orthogonal IL-2 can be fused to the Fc domain of IgG, albumin, or other molecules to extend its half-life, e.g. by pegylation, glycosylation, and the like as known in the art. In some embodiments the orthogonal cytokine is conjugated to a polyethylene glycol molecules or “PEGylated.” The molecular weight of the PEG conjugated to the orthogonal cytokine ligand include but are not limited to PEGs having molecular weights between 5 kDa and 80 kDa, in some embodiments the PEG has a molecular weight of approximately 5 kDa, in some embodiments the PEG has a molecular weight of approximately 10 kDa, in some embodiments the PEG has a molecular weight of approximately 20 kDa, in some embodiments the PEG has a molecular weight of approximately 30 kDa, in some embodiments the PEG has a molecular weight of approximately 40 kDa, in some embodiments the PEG has a molecular weight of approximately 50 kDa, in some embodiments the PEG has a molecular weight of approximately 60 kDa in some embodiments the PEG has a molecular weight of approximately 80 kDa. In some embodiments, the molecular mass is from about 5 kDa to about 80 kDa, from about 5 kDa to about 60 kDa, from about 5 kDa to about 40 kDa, from about 5 kDa to about 20 kDa. In preferred embodiments, (where naming of polypeptides is made with reference to Table 1), the orthogonal ligand is a pegylated form of 1A1, a pegylated form of 1C7, a pegylated form of SQVLKA, and/or a pegylated form of SQVLqA, in each instance the PEG having a molecular weight of approximately 5 kDa, alternatively 10 kDa, 20 kDa, alternatively 30 kDa, alternatively 40 kDa alternatively 40 kDa, alternatively 50 kDa, alternatively 30 kDa. The PEG conjugated to the polypeptide sequence may be linear or branched. The PEG may be attached directly to the orthogonal polypeptide or via a linker molecule. The processes and chemical reactions necessary to achieve PEGylation of biological compounds is well known in the art.

Orthogonal IL-2 can be acetylated at the N-terminus, using methods known in the art, e.g. by enzymatic reaction with N-terminal acetyltransferase and, for example, acetyl CoA. Orthogonal IL-2 can be acetylated at one or more lysine residues, e.g. by enzymatic reaction with a lysine acetyltransferase. See, for example Choudhary et al. (2009). Science. 325 (5942): 834-840.

Fc-fusion can also endow alternative Fc receptor mediated properties in vivo. The “Fc region” can be a naturally occurring or synthetic polypeptide that is homologous to an IgG C-terminal domain produced by digestion of IgG with papain. IgG Fc has a molecular weight of approximately 50 kDa. The ortholog IL-2 polypeptides can include the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the polypeptides; as described further below, native activity is not necessary or desired in all cases.

In other embodiments, an orthogonal polypeptide can comprise polypeptide that functions as an antigenic tag, such as a FLAG sequence. FLAG sequences are recognized by biotinylated, highly specific, anti-FLAG antibodies, as described herein (see also Blanar et al., Science 256:1014, 1992; LeClair et al., Proc. Natl. Acad. Sci. USA 89:8145, 1992). In some embodiments, the chimeric polypeptide further comprises a C-terminal c-myc epitope tag.

As described above, the orthogonal proteins of the invention may exist as a part of a chimeric polypeptide. In addition to, or in place of, the heterologous polypeptides described above, a nucleic acid molecule of the invention can contain sequences encoding a “marker” or “reporter.” Examples of marker or reporter genes include β-lactamase, chloramphenicol acetyltransferase (CAT), adenosine deaminase (ADA), aminoglycoside phosphotransferase (neo1, G418r), dihydrofolate reductase (DHFR), hygromycin-B-hosphotransferase (HPH), thymidine kinase (TK), lacz (encoding β-galactosidase), and xanthine guanine phosphoribosyltransferase (XGPRT). As with many of the standard procedures associated with the practice of the invention, skilled artisans will be aware of additional useful reagents, for example, of additional sequences that can serve the function of a marker or reporter.

Orthogonal cytokines and receptors may also include conservative modifications and substitutions at other positions of the cytokine (e.g. positions other than those involved in the orthogonal engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: ala, pro, gly, gin, asn, ser, thr; Group II: cys, ser, tyr, thr; Group III: val, ile, leu, met, ala, phe; Group IV: lys, arg, his; Group V: phe, tyr, trp, his; and Group VI: asp, glu. In each instance, the introduction of additional modifications may be evaluated to minimize any increase in antigenicity of the modified polypeptide in the organism to which the modified polypeptide is to be administered.

The term “T cells” refers to mammalian immune effector cells that may be characterized by expression of CD3 and/or T cell antigen receptor, which cells may be engineered to express an orthogonal cytokine receptor. In some embodiments the T cells are selected from naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, natural T_{Reg}, inducible T_{Reg}; memory T cells, e.g. central memory T cells, effector memory T cells, NKT cells, γδ T cells.

In one embodiment of the invention the T-cell expressing the orthogonal receptor is a T-cell which has been modified to surface express a chimeric antigen receptor (a ‘CAR-T’ cell). As used herein, the terms “chimeric antigen receptor T-cell” and “CAR-T cell” are used interchangeably to refer to a T-cell that has been recombinantly modified to express a chimeric antigen receptor. As used herein, a CAR-T cell may be engineered to express an orthogonal IL-2Rβ polypeptide. As used herein, the terms “chimeric antigen receptor” and “CAR” are used interchangeably to refer to a polypeptide comprising multiple functional domains arranged from amino to carboxy terminus in the sequence: (a) an antigen binding domain (ABD), (b) a transmembrane domain (TD); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface. CARs useful in the practice of the present invention are prepared in accordance with principles well known in the art. See e.g., Eshhaar et al. U.S. Pat. No. 7,741,465 B1 issued Jun. 22, 2010; Sadelain, et al (2013) Cancer Discovery 3 (4): 388-398; Jensen and Riddell (2015) Current Opinions in Immunology 33:9-15; Gross, et al. (1989) PNAS (USA) 86 (24): 10024-10028; Curran, et al. (2012) J Gene Med 14 (6): 405-15. Examples of commercially available CAR-T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis).

As used herein, the term antigen binding domain (ABD) refers to a polypeptide that specifically binds to an antigen expressed on the surface of a target cell. The ABD may be any polypeptide that specifically binds to one or more antigens expressed on the surface of a target cell. In certain embodiments, the target cell antigen is a tumor antigen. Examples of tumor antigens that may be targeted by the ABD of the CAR include one or more antigens selected from the group including, but not limited to, the CD19, CD20, HER2, NY-ESO-1, MUC1, CD123, FLT3, B7-H3, CD33, IL1RAP, CLL1 (CLEC12A) PSA, CEA, VEGF, VEGF-R2, CD22, ROR1, mesothelin, c-Met, Glycolipid F77, FAP, EGFRvIII, MAGE A3, 5T4, WT1, KG2D ligand, a folate receptor (FRa), and Wnt1 antigens.

In one embodiment, the ABD is a single chain Fv (ScFv). An ScFv is a polypeptide comprised of the variable regions of the immunoglobulin heavy and light chain of an antibody covalently connected by a peptide linker (Bird, et al. (1988) Science 242:423-426; Huston, et al. (1988) PNAS (USA) 85:5879-5883; S-z Hu, et al. (1996) Cancer Research, 56, 3055-3061. The generation of ScFvs based on monoclonal antibody sequences is well known in the art. See, e.g. The Protein Protocols Handbook, John M. Walker, Ed. (2002) Humana Press Section 150 “Bacterial Expression, Purification and Characterization of Single-Chain Antibodies” Kipriyanov, S. Antibodies used in the preparation of scFvs may be optimized to select for those molecules which possess particular desirable characteristics (e.g. enhanced affinity) through techniques well known in the art such as phage display and directed evolution. In some embodiments, the ABD comprises an anti-CD19 scFv, an anti-PSA scFv, an anti-HER2 scFv, an anti-CEA scFv, an anti-EGFR scFv, an anti-EGFRvIII scFv, an anti-NY-ESO-1 scFv, an anti-MAGE scFv, an anti-5T4 scFv, or an anti-Wnt1 scFv. In another embodiment, the ABD is a single domain antibody obtained through immunization of a camel or llama with a target cell derived antigen, in particular a tumor antigen. See, e.g. Muyldermans, S. (2001) Reviews in Molecular Biotechnology 74:277-302. Alternatively, the ABD may be generated wholly synthetically through the generation of peptide libraries and isolating compounds having the desired target cell antigen binding properties in substantial accordance with the teachings or Wigler, et al. U.S. Pat. No. 6,303,313 B1 issued Nov. 12, 1999; Knappik, et al., U.S. Pat. No. 6,696,248 B1 issued Feb. 24, 2004, Binz, et al. (2005) Nature Biotechnology 23:1257-1268, and Bradbury, et al. (2011) Nature Biotechnology 29:245-254.

The ABD may have affinity for more than one target antigen. For example, an ABD of the present invention may comprise chimeric bispecific binding members, i.e. have capable of providing for specific binding to a first target cell expressed antigen and a second target cell expressed antigen. Non-limiting examples of chimeric bispecific binding members include bispecific antibodies, bispecific conjugated monoclonal antibodies (mab)₂, bispecific antibody fragments (e.g., F(ab)₂, bispecific scFv, bispecific diabodies, single chain bispecific diabodies, etc.), bispecific T cell engagers (BITE), bispecific conjugated single domain antibodies, micabodies and mutants thereof, and the like. Non-limiting examples of chimeric bispecific binding members also include those chimeric bispecific agents described in Kontermann (2012) MAbs. 4 (2): 182-197; Stamova et al. (2012) Antibodies, 1 (2), 172-198; Farhadfar et al. (2016) Leuk Res. 49:13-21; Benjamin et al. Ther Adv Hematol. (2016) 7 (3): 142-56; Kiefer et al. Immunol Rev. (2016) 270 (1): 178-92; Fan et al. (2015) J Hematol Oncol. 8:130; May et al. (2016) Am J Health Syst Pharm. 73 (1): e6-e13. In some embodiments, the chimeric bispecific binding member is a bivalent single chain polypeptides. See, e.g. Thirion, et al. (1996) European J. of Cancer Prevention 5 (6): 507-511; DeKruif and Logenberg (1996) J. Biol. Chem 271 (13) 7630-7634; and Kay, et al. United States Patent Application Publication Number 2015/0315566 published Nov. 5, 2015. In some instances, a chimeric bispecific binding member may be a bispecific T cell engager (BiTE). A BITE is generally made by fusing a specific binding member (e.g., a scFv) that binds an antigen to a specific binding member (e.g., a scFv) with a second binding domain specific for a T cell molecule such as CD3. In some instances, a chimeric bispecific binding member may be a CAR T cell adapter. As used herein, by “CAR T cell adapter” is meant an expressed bispecific polypeptide that binds the antigen recognition domain of a CAR and redirects the CAR to a second antigen. Generally, a CAR T cell adapter will have to binding regions, one specific for an epitope on the CAR to which it is directed and a second epitope directed to a binding partner which, when bound, transduces the binding signal activating the CAR. Useful CAR T cell adapters include but are not limited to e.g., those described in Kim et al. (2015) J Am Chem Soc. 137 (8): 2832-5; Ma et al. (2016) Proc Natl Acad Sci USA. 113 (4): E450-8 and Cao et al. (2016) Angew Chem Int Ed Engl. 55 (26): 7520-4.

In some embodiments, a linker polypeptide molecule is optionally incorporated into the CAR between the antigen binding domain and the transmembrane domain to facilitate antigen binding. Moritz and Groner (1995) Gene Therapy 2 (8) 539-546. In one embodiment, the linker is the hinge region from an immunoglobulin, e.g. the hinge from any one of IgG1, IgG2a, IgG2b, IgG3, IgG4, particularly the human protein sequences. Alternatives include the CH2CH3 region of immunoglobulin and portions of CD3. In those instances where the ABD is an scFv, an IgG hinge may be employed. In some embodiments the linker comprises the amino acid sequence (G4S) n where n is 1, 2, 3, 4, 5, etc., and in some embodiments n is 3.

CARs useful in the practice of the present invention further comprise a transmembrane domain joining the ABD (or linker, if employed) to the intracellular cytoplasmic domain of the CAR. The transmembrane domain is comprised of any polypeptide sequence which is thermodynamically stable in a eukaryotic cell membrane. The transmembrane spanning domain may be derived from the transmembrane domain of a naturally occurring membrane spanning protein or may be synthetic. In designing synthetic transmembrane domains, amino acids favoring alpha-helical structures are preferred. Transmembrane domains useful in construction of CARs are comprised of approximately 10, 11, 12, 13, 14, 15, 16, 17, 18, 19, 20, 21, 22, 22, 23, or 24 amino acids favoring the formation having an alpha-helical secondary structure. Amino acids having a to favor alpha-helical conformations are well known in the art. See, e.g Pace, et al. (1998) Biophysical Journal 75:422-427. Amino acids that are particularly favored in alpha helical conformations include methionine, alanine, leucine, glutamate, and lysine. In some embodiments, the CAR transmembrane domain may be derived from the transmembrane domain from type I membrane spanning proteins, such as CD3Z, CD4, CD8, CD28, etc.

The cytoplasmic domain of the CAR polypeptide comprises one or more intracellular signal domains. In one embodiment, the intracellular signal domains comprise the cytoplasmic sequences of the T-cell receptor (TCR) and co-receptors that initiate signal transduction following antigen receptor engagement and functional derivatives and sub-fragments thereof. A cytoplasmic signaling domain, such as those derived from the T cell receptor ζ-chain, is employed as part of the CAR in order to produce stimulatory signals for T lymphocyte proliferation and effector function following engagement of the chimeric receptor with the target antigen. Examples of cytoplasmic signaling domains include but are not limited to the cytoplasmic domain of CD27, the cytoplasmic domain S of CD28, the cytoplasmic domain of CD137 (also referred to as 4-1BB and TNFRSF9), the cytoplasmic domain of CD278 (also referred to as ICOS), p110α, β, or δ catalytic subunit of PI3 kinase, the human CD3 ζ-chain, cytoplasmic domain of CD134 (also referred to as OX40 and TNFRSF4), FcεR1γ and β chains, MB1 (Igα) chain, B29 (Igβ) chain, etc.), CD3 polypeptides (δ, Δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28.

In some embodiments, the CAR may also provide a co-stimulatory domain. The term “co-stimulatory domain”, refers to a stimulatory domain, typically an endodomain, of a CAR that provides a secondary non-specific activation mechanism through which a primary specific stimulation is propagated. The co-stimulatory domain refers to the portion of the CAR which enhances the proliferation, survival or development of memory cells. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved have been described in Chen & Flies. (2013) Nat Rev Immunol 13 (4): 227-42. In some embodiments of the present disclosure, the CSD comprises one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or combinations thereof.

CARs are often referred to as first, second, third or fourth generation. The term first-generation CAR refers to a CAR wherein the cytoplasmic domain transmits the signal from antigen binding through only a single signaling domain, for example a signaling domain derived from the high-affinity receptor for IgE FcεRIγ. or the CD3ζ chain. The domain contains one or three immunoreceptor tyrosine-based activating motif(s) [ITAM(s)] for antigen-dependent T-cell activation. The ITAM-based activating signal endows T-cells with the ability to lyse the target tumor cells and secret cytokines in response to antigen binding. Second-generation CARs include a co-stimulatory signal in addition to the CD3ζ signal. Coincidental delivery of the delivered co-stimulatory signal enhances cytokine secretion and antitumor activity induced by CAR-transduced T-cells. The co-stimulatory domain is usually be membrane proximal relative to the CD35 domain. Third-generation CARs include a tripartite signaling domain, comprising for example a CD28, CD3ζ, OX40 or 4-1BB signaling region. In fourth generation, or “armored car” CAR T-cells are further gene modified to express or block molecules and/or receptors to enhance immune activity.

Examples of intracellular signaling domains comprising may be incorporated into the CAR of the present invention include (amino to carboxy): CD3ζ; CD28-41BB-CD3ζ; CD28-OX40-CD3ζ; CD28-41BB-CD3ζ; 41BB-CD-28-CD3ζ and 41BB-CD3ζ.

The term CAR includes CAR variants including but not limited split CARs, ON-switch CARS, bispecific or tandem CARs, inhibitory CARs (iCARs) and induced pluripotent stem (IPS) CAR-T cells.

The term “Split CARs” refers to CARs wherein the extracellular portion, the ABD and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US1996/017060, US2013/063083; Fedorov et al. Sci Transl Med (2013); 5 (215): 215ra172; Glienke et al. Front Pharmacol (2015) 6:21; Kakarla & Gottschalk 52 Cancer J (2014) 20 (2): 151-5; Riddell et al. Cancer J (2014) 20 (2): 141-4; Pegram et al. Cancer J (2014) 20 (2): 127-33; Cheadle et al. Immunol Rev (2014) 257 (1): 91-106; Barrett et al. Annu Rev Med (2014) 65:333-47; Sadelain et al. Cancer Discov (2013) 3 (4): 388-98; Cartellieri et al., J Biomed Biotechnol (2010) 956304; the disclosures of which are incorporated herein by reference in their entirety.

The term “bispecific or tandem CARs” refers to CARs which include a secondary CAR binding domain that can either amplify or inhibit the activity of a primary CAR.

The term “inhibitory chimeric antigen receptors” or “iCARs” are used interchangeably herein to refer to a CAR where binding iCARs use the dual antigen targeting to shut down the activation of an active CAR through the engagement of a second suppressive receptor equipped with inhibitory signaling domains of a secondary CAR binding domain results in inhibition of primary CAR activation. Inhibitory CARs (iCARs) are designed to regulate CAR-T cells activity through inhibitory receptors signaling modules activation. This approach combines the activity of two CARs, one of which generates dominant negative signals limiting the responses of CAR-T cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes, leading to less cytokine production, less efficient target cell lysis, and altered lymphocyte motility.

The term “tandem CAR” or “TanCAR” refers to CARs which mediate bispecific activation of T cells through the engagement of two chimeric receptors designed to deliver stimulatory or costimulatory signals in response to an independent engagement of two different tumor associated antigens.

Typically, the chimeric antigen receptor T-cells (CAR-T cells) are T-cells which have been recombinantly modified by transduction with an expression vector encoding a CAR in substantial accordance with the teaching above.

Cells may be prepared using the patient's own T-cells for engineering. Consequently, the population of the cells to be administered is to the subject is necessarily variable. Additionally, since the CAR-T cell agent is variable, the response to such agents can vary and thus involves the ongoing monitoring and management of therapy related toxicities which are managed with a course of pharmacologic immunosuppression or B cell depletion prior to the administration of the CAR-T cell treatment. Examples of such immunosuppressive regimens including systemic corticosteroids (e.g., methylprednisolone). Therapies for B cell depletion include intravenous immunoglobulin (IVIG) by established clinical dosing guidelines to restore normal levels of serum immunoglobulin levels. In some embodiments, prior to administration of the CAR-T cell therapy of the present invention, the subject may optionally be subjected to a lymphodepleting regimen. One example of a such lymphodepleting regimen consists of the administration to the subject of fludarabine (30 mg/m²intravenous [IV] daily for 4 days) and cyclophosphamide (500 mg/m² IV daily for 2 days starting with the first dose of fludarabine).

T-cells useful for engineering with the constructs described herein include naïve T-cells, central memory T-cells, effector memory T-cells or combination thereof. T cells for engineering as described above are collected from a subject or a donor may be separated from a mixture of cells by techniques that enrich for desired cells or may be engineered and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. Techniques for affinity separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS). The collected and optionally enriched cell population may be used immediately for genetic modification, or may be frozen at liquid nitrogen temperatures and stored, being thawed and capable of being reused. The cells will usually be stored in 10% DMSO, 50% FCS, 40% RPMI 1640 medium.

In some embodiments, the engineered cells comprise a complex mixture of immune cells, e.g., tumor infiltrating lymphocytes (TILs) isolated from an individual in need of treatment. See, for example, Yang and Rosenberg (2016) Adv Immunol. 130:279-94, “Adoptive T Cell Therapy for Cancer; Feldman et al (2015) Semin Oncol. 42 (4): 626-39 “Adoptive Cell Therapy-Tumor-Infiltrating Lymphocytes, T-Cell Receptors, and Chimeric Antigen Receptors”; Clinical Trial NCT01174121, “Immunotherapy Using Tumor Infiltrating Lymphocytes for Patients With Metastatic Cancer”; Tran et al. (2014) Science 344 (6184) 641-645, “Cancer immunotherapy based on mutation-specific CD4+ T cells in a patient with epithelial cancer”.

In some embodiments, an engineered T cell is allogeneic with respect to the individual that is treated, e.g. see clinical trials NCT03121625; NCT03016377; NCT02476734; NCT02746952; NCT02808442. See for review Graham et al. (2018) Cells. 7 (10) E155. In some embodiments an allogeneic engineered T cell is fully HLA matched. However not all patients have a fully matched donor and a cellular product suitable for all patients independent of HLA type provides an alternative. A universal ‘off the shelf’ T cell product provides advantages in uniformity of harvest and manufacture.

T cells for engineering as described above collected from a subject or a donor may be separated from a mixture of cells by techniques that enrich for desired cells or may be engineered and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. Techniques for affinity separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like. The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS). The collected and optionally enriched cell population may be used immediately for genetic modification, or may be frozen at liquid nitrogen temperatures and stored, being thawed and capable of being reused. The cells will usually be stored in 10% DMSO, 50% FCS, 40% RPMI 1640 medium. The engineered cells may be infused to the subject in any physiologically acceptable medium by any convenient route of administration, normally intravascularly, although they may also be introduced by other routes, where the cells may find an appropriate site for growth. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection.

The allogeneic T cells used in the practice of the present invention may be genetically modified to reduce graft versus host disease. For example the engineered cells of the present invention may be TCRαβ receptor knock-outs achieved by gene editing techniques. TCRαβ is a heterodimer and both alpha and beta chains need to be present for it to be expressed. A single gene codes for the alpha chain (TRAC), whereas there are 2 genes coding for the beta chain, therefore TRAC loci KO has been deleted for this purpose. A number of different approaches have been used to accomplish this deletion, e.g. CRISPR/Cas9; meganuclease; engineered I-Crel homing endonuclease, etc. See, for example, Eyquem et al. (2017) Nature 543:113-117, in which the TRAC coding sequence is replaced by a CAR coding sequence; and Georgiadis et al. (2018) Mol. Ther. 26:1215-1227, which linked CAR expression with TRAC disruption by clustered regularly interspaced short palindromic repeats (CRISPR)/Cas9 without directly incorporating the CAR into the TRAC loci. An alternative strategy to prevent GVHD modifies T cells to express an inhibitor of TCRαβ signaling, for example using a truncated form of CD34 as a TCR inhibitory molecule.

The preparation of T-cells useful in the practice of the present invention is achieved by transforming isolated T-cells with an expression vector comprising a nucleic acid sequence encoding the orthogonal receptor; optionally in combination with a nucleic acid sequence encoding a CAR polypeptide described above. The nucleic acid sequences encoding a CAR and an orthogonal receptor may each be provided on separate expression vectors, each nucleic acid sequence being operably linked to one or more expression control elements to achieve expression of the CAR and orthogonal receptor in the target cell, the vectors being co-transfected into the target cell. Alternatively, the nucleic acid sequences encoding the CAR and the orthogonal receptor may each be provided on a single vector each nucleic acid sequence under the control of one or more expression control elements to achieve expression of the associated nucleic acid sequence. Alternatively, both nucleic acid sequences may be under the control of a single promoter with intervening or downstream control elements that facilitate co-expression of the two sequences from the vector.

Ex vivo T-cell activation may be achieved by procedures well established in the art including cell-based T-cell activation, antibody-based activation or activation using a variety of bead-based activation reagents. Cell-based T-cell activation may be achieved by exposure of the T-cells to antigen presenting cells, such as dendritic cells or artificial antigen presenting cells such as irradiated K562 cells. Antibody based activation of T-cell surface CD3 molecules with soluble anti-CD3 monoclonal antibodies also supports T-cell activation in the presence of IL-2.

Generally, the T-cells of the invention are expanded by culturing the cells in contact with a surface providing an agent that stimulates a CD3 TCR complex associated signal (e.g., an anti-CD3 antibody) and an agent that stimulates a co-stimulatory molecule on the surface of the T-cells (e.g an anti-CD28 antibody). Bead-based T-cell activation has gained acceptance in the art for the preparation of CAR-T cells for clinical use. Bead-based activation of T-cells may be achieved using commercially available T-cell activation reagents including but not limited to the Invitrogen® CTS Dynabeads® CD3/28 (Life Technologies, Inc. Carlsbad CA) or Miltenyi MACS® GMP ExpAct Treg beads or Miltenyi MACS GMP TransAct™ CD3/28 beads (Miltenyi Biotec, Inc.). Conditions appropriate for T-cell culture are well known in the art. Lin, et al. (2009) Cytotherapy 11 (7): 912-922; Smith, et al. (2015) Clinical & Translational Immunology 4: e31 published online 16 Jan. 2015. The target cells are maintained under conditions necessary to support growth, for example, an appropriate temperature (e.g., 37° C.) and atmosphere (e.g., air plus 5% CO₂).

When the orthogonal receptor or the orthogonal receptor expressing CAR-T cell is a growth factor receptor, the orthogonal receptor expressing CAR-T cells may also be selectively expanded from the background or mixed population of transduced and non-transduced cells through the use of a ligand for the orthogonal receptor. In one embodiment, the orthogonal receptor is an orthogonal IL-2 receptor and the orthogonal IL-2 compound useful in the expansion of such cells is an orthogonal IL-2 selected from the group provided in Table 1.

In the present methods, an orthogonal protein, particularly the orthogonal cytokine, may be produced by recombinant methods. The orthogonal receptor may be introduced on an expression vector into the cell to be engineered. DNA encoding an orthogonal protein may be obtained from various sources as designed during the engineering process.

Amino acid sequence variants are prepared by introducing appropriate nucleotide changes into the coding sequence, as described herein. Such variants represent insertions, substitutions, and/or specified deletions of, residues as noted. Any combination of insertion, substitution, and/or specified deletion is made to arrive at the final construct, provided that the final construct possesses the desired biological activity as defined herein.

To achieve expression of the recombinant protein, a nucleic acid encoding an orthogonal protein (and/or CAR) is inserted into a replicable vector for expression. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

Expression vectors for expression of the orthogonal receptor and optionally CAR in the T-cell may be viral vectors or non-viral vectors. Plasmids are examples of non-viral vectors. In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

In one embodiment, a non-viral vector may be provided in a non-viral delivery system. Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are well known in the art including lipidic vector systems (Lee et al. (1997) Crit Rev Ther Drug Carrier Syst. 14:173-206); polymer coated liposomes (Marin et al., U.S. Pat. No. 5,213,804, issued May 25, 1993; Woodle, et al., U.S. Pat. No. 5,013,556, issued May 7, 1991); cationic liposomes (Epand et al., U.S. Pat. No. 5,283,185, issued Feb. 1, 1994; Jessee, J. A., U.S. Pat. No. 5,578,475, issued Nov. 26, 1996; Rose et al, U.S. Pat. No. 5,279,833, issued Jan. 18, 1994; Gebeyehu et al., U.S. Pat. No. 5,334,761, issued Aug. 2, 1994).

In another embodiment, the expression vector may be a viral vector. When a viral vector system is to be employed for CAR and expression of the orthogonal receptor, retroviral or lentiviral expression vectors are preferred. In particular, the viral vector is a gamma retrovirus (. (Pule, et al. (2008) Nature Medicine 14 (11): 1264-1270), self-inactivating lentiviral vectors (June, et al. (2009) Nat Rev Immunol 9 (10): 704-716) and retroviral vectors as described in Naldini, et al. (1996) Science 272:263-267; Naldini, et al. (1996) Proc. Natl. Acad. Sci. USA Vol. 93, pp. 11382-11388; Dull, et al. (1998) J. Virology 72 (11): 8463-8471; Milone, et al. (2009) 17 (8): 1453-1464; Kingsman, et al. U.S. Pat. No. 6,096,538 issued Aug. 1, 2000 and Kingsman, et al. U.S. Pat. No. 6,924,123 issued Aug. 2, 2005. In one embodiment of the invention, the CAR expression vector is a Lentivector® lentiviral vector available from Oxford Biomedica.

Transduction of T-cells with an expression vector may be accomplished using techniques well known in the art including but not limited co-incubation with host T-cells with viral vectors, electroporation, and/or chemically enhanced delivery.

An orthogonal protein may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In mammalian cell expression the native signal sequence may be used, or other mammalian signal sequences may be suitable, such as signal sequences from secreted polypeptides of the same or related species, as well as viral secretory leaders, for example, the herpes simplex gD signal.

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors will contain a promoter that is recognized by the host organism and is operably linked to an orthogonal protein coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter.

Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning or expressing the DNA in the vectors herein are the prokaryote, yeast, or higher eukaryote cells described above. Examples of useful mammalian host cell lines are mouse L cells (L-M [TK-], ATCC #CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells, including engineered T cells, can be transfected with the above-described expression vectors for orthogonal IL-2, or IL-2R expression. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

Recombinantly produced orthogonal polypeptides can be recovered from the culture medium as a secreted polypeptide, although it can also be recovered from host cell lysates. A protease inhibitor, such as phenyl methyl sulfonyl fluoride (PMSF) also may be useful to inhibit proteolytic degradation during purification, and antibiotics may be included to prevent the growth of adventitious contaminants. Various purification steps are known in the art and find use, e.g. affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural biospecific binding of one molecular species to separate and purify a second species from a mixture. Antibodies are commonly used in affinity chromatography. Size selection steps may also be used, e.g. gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column. Also of interest is cation exchange chromatography.

The orthogonal cytokine composition may be concentrated, filtered, dialyzed, etc., using methods known in the art. For therapeutic applications, the cytokines can be administered to a mammal comprising the appropriate engineered orthogonal receptor. Administration may be intravenous, as a bolus or by continuous infusion over a period of time. Alternative routes of administration include intramuscular, intraperitoneal, intra-cerobrospinal, subcutaneous, intra-articular, intrasynovial, intrathecal, oral, topical, or inhalation routes. The orthogonal cytokines also are suitably administered by intratumoral, peritumoral, intralesional, or perilesional routes or to the lymph, to exert local as well as systemic therapeutic effects.

Such dosage forms encompass physiologically acceptable carriers that are inherently non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of polypeptides include polysaccharides such as sodium carboxymethylcellulose or methylcellulose, polyvinylpyrrolidone, polyacrylates, polyoxyethylene-polyoxypropylene-block polymers, PEG, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. The polypeptide will typically be formulated in such vehicles at a concentration of about 0.1 μg/ml to 100 μg/ml.

In the event the ortholog IL-2 polypeptides of the disclosure are “substantially pure,” they can be at least about 60% by weight (dry weight) the polypeptide of interest, for example, a polypeptide containing the ortholog IL-2 amino acid sequence. For example, the polypeptide can be at least about 75%, about 80%, about 85%, about 90%, about 95% or about 99%, by weight, the polypeptide of interest. Purity can be measured by any appropriate standard method, for example, column chromatography, polyacrylamide gel electrophoresis, or HPLC analysis.

In another embodiment of the invention, an article of manufacture containing materials useful for the treatment of the conditions described above is provided. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). The active agent in the composition is the orthogonal cytokine. The label on, or associated with, the container indicates that the composition is used for treating the condition of choice. Further container(s) may be provided with the article of manufacture which may hold, for example, a pharmaceutically-acceptable buffer, such as phosphate-buffered saline, Ringer's solution or dextrose solution. The article of manufacture may further include other materials desirable from a commercial and user standpoint, including other buffers, diluents, filters, needles, syringes, and package inserts with instructions for use.

As used herein, the terms “cancer” (or “cancerous”), “hyperproliferative,” and “neoplastic” to refer to cells having the capacity for autonomous growth (e.g., an abnormal state or condition characterized by rapidly proliferating cell growth). Hyperproliferative and neoplastic disease states may be categorized as pathologic (e.g., characterizing or constituting a disease state), or they may be categorized as non-pathologic (e.g., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures.

Examples of tumor cells include but are not limited to AML, ALL, CML, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, Hodgkin's lymphoma, Kaposi's sarcoma, kidney cancer, laryngeal and hypopharyngeal cancer, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia. Any cancer, where the cancer cells exhibit increased expression of CD47 compared to non-cancer cells, is a suitable cancer to be treated by the subject methods and compositions.

The compositions and method of the present invention may be combined with additional therapeutic agents. For example, when the disease, disorder or condition to be treated is a neoplastic disease (e.g. cancer) the methods of the present in ivention may be combined with conventional chemotherapeutic agents or other biological anti-cancer drugs such as checkpoint inhibitors (e.g. PD1 or PDL1 inhibitors) or therapeutic monoclonal antibodies (e.g Avastin, Herceptin).

Examples of chemical agents identified in the art as useful in the treatment of neoplastic disease, include without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Targeted therapeutics that can be administered in combination may include, without limitation, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade), Jakafi (ruxolitinib); Janus kinase inhibitors, such as tofacitinib; ALK inhibitors, such as crizotinib; Bcl-2 inhibitors, such as obatoclax, venclexta, and gossypol; FLT3 inhibitors, such as midostaurin (Rydapt), IDH inhibitors, such as AG-221, PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys (6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

Examples of biological agents identified in the art as useful in the treatment of neoplastic disease, include without limitation, cytokines or cytokine antagonists such as IL-12, INFα, or anti-epidermal growth factor receptor, radiotherapy, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, cyclooxygenase-2 (COX-2) inhibitors, steroids, TNF antagonists (e.g., Remicade® and Enbrel®), interferon-31a (Avonex®), and interferon-α1b (Betaseron®) as well as combinations of one or more of the foregoing as practiced in known chemotherapeutic treatment regimens readily appreciated by the skilled clinician in the art.

Tumor specific monoclonal antibodies that can be administered in combination with an anti-CD93 ABD polypeptide or engineered cell may include, without limitation, Rituximab (marketed as MabThera or Rituxan), Alemtuzumab, Panitumumab, Ipilimumab (Yervoy), etc.

In some embodiments the compositions and methods of the present invention may be combined with immune checkpoint therapy. Examples of immune checkpoint therapies include inhibitors of the binding of PD1 to PDL1 and/or PDL2. PD1 to PDL1 and/or PDL2 inhibitors are well known in the art. Examples of commercially available monoclonal antibodies that interfere with the binding of PD1 to PDL1 and/or PDL2 include nivolumab (Opdivo®, BMS-936558, MDX1106, commercially available from BristolMyers Squibb, Princeton NJ), pembrolizumab (Keytruda®MK-3475, lambrolizumab, commercially available from Merck and Company, Kenilworth NJ), and atezolizumab (Tecentriq®, Genentech/Roche, South San Francisco CA). Additional examples of PD1 inhibitory antibodies include but are not limited to durvalumab (MEDI4736, Medimmune/AstraZeneca), pidilizumab (CT-011, CureTech), PDR001 (Novartis), BMS-936559 (MDX1105, Bristol Myers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011. Additionally, small molecule PD1 to PDL1 and/or PDL2 inhibitors are known in the art. See, e.g. Sasikumar, et al as WO2016142833A1 and Sasikumar, et al. WO2016142886A2, BMS-1166 and BMS-1001 (Skalniak, et al (2017) Oncotarget 8 (42): 72167-72181).

In other embodiments the methods of the invention are used in the treatment of infection. As used herein, the term “infection” refers to any state in at least one cell of an organism (i.e., a subject) is infected by an infectious agent (e.g., a subject has an intracellular pathogen infection, e.g., a chronic intracellular pathogen infection). As used herein, the term “infectious agent” refers to a foreign biological entity (i.e. a pathogen) that induces increased CD47 expression in at least one cell of the infected organism. For example, infectious agents include, but are not limited to bacteria, viruses, protozoans, and fungi. Intracellular pathogens are of particular interest. Infectious diseases are disorders caused by infectious agents. Some infectious agents cause no recognizable symptoms or disease under certain conditions, but have the potential to cause symptoms or disease under changed conditions. The subject methods can be used in the treatment of chronic pathogen infections, for example including but not limited to viral infections, e.g. retrovirus, lentivirus, hepadna virus, herpes viruses, pox viruses, human papilloma viruses, etc.; intracellular bacterial infections, e.g. Mycobacterium, Chlamydophila, Ehrlichia, Rickettsia, Brucella, Legionella, Francisella, Listeria, Coxiella, Neisseria, Salmonella, Yersinia sp, Helicobacter pylori etc.; and intracellular protozoan pathogens, e.g. Plasmodium sp, Trypanosoma sp., Giardia sp., Toxoplasma sp., Leishmania sp., etc.

Treatment may be combined with other active agents. Classes of antibiotics include penicillins, e.g. penicillin G, penicillin V, methicillin, oxacillin, carbenicillin, nafcillin, ampicillin, etc.; penicillins in combination with α-lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. Cytokines may also be included, e.g. interferon γ, tumor necrosis factor α, interleukin 12, etc. Antiviral agents, e.g. acyclovir, gancyclovir, etc., may also be used in treatment.

In yet other embodiments, regulatory T cells are engineered for the treatment of autoimmune disease. The spectrum of inflammatory diseases and diseases associated with inflammation is broad and includes autoimmune diseases such rheumatoid arthritis (RA), systemic lupus erythematosus (SLE), multiple sclerosis (MS), and autoimmune hepatitis; insulin dependent diabetes mellitus, degenerative diseases such as osteoarthritis (OA), Alzheimer's disease (AD), and macular degeneration.

Many, if not most, autoimmune and inflammatory diseases involve multiple types of T cells, e.g. T_{H}1, T_{H}2, T_{H}17, and the like. Autoimmune diseases are characterized by T and B lymphocytes that aberrantly target self-proteins, -polypeptides, -peptides, and/or other self-molecules causing injury and or malfunction of an organ, tissue, or cell-type within the body (for example, pancreas, brain, thyroid or gastrointestinal tract) to cause the clinical manifestations of the disease. Autoimmune diseases include diseases that affect specific tissues as well as diseases that can affect multiple tissues, which can depend, in part on whether the responses are directed to an antigen confined to a particular tissue or to an antigen that is widely distributed in the body.

Engineered orthogonal cytokine receptor/ligand pairs, and methods of use thereof, are provided. An engineered (orthogonal) cytokine specifically binds to a counterpart engineered (orthogonal) receptor. Upon binding, the orthogonal receptor activates signaling that is transduced through native cellular elements to provide for a biological activity that mimics that native response, but which is specific to an engineered cell expressing the orthogonal receptor. The orthogonal receptor exhibits significantly reduced binding to the endogenous counterpart cytokine, including the native counterpart of the orthogonal cytokine, while the orthogonal cytokine exhibits significantly reduced binding to any endogenous receptors, including the native counterpart of the orthogonal receptor. In some embodiments, the affinity of the orthogonal cytokine for the orthogonal receptor is comparable to the affinity of the native cytokine for the native receptor.

The orthogonal cytokine and receptor pair may be selected from any cytokine of interest. The process for engineering an orthogonal cytokine receptor pair may comprise the steps of (a) engineering amino acid changes into a native receptor to disrupt binding to the native cytokine; (b) engineering amino acid changes into the native cytokine at contact residues for receptor binding, (c) selecting for cytokine orthologs that bind to the orthogonal receptor; (d) discarding ortholog cytokines that bind to the native receptor, or (e) selecting for receptor orthologs that bind the ortholog cytokine; (f) discarding ortholog receptors that bind to the native cytokine. In preferred embodiments, knowledge of the structure of the cytokine/receptor complex is used to select amino acid positions for site-directed or error prone mutagenesis. Conveniently a yeast display system can be used for the selection process, although other display and selection methods are also useful.

In some cases, amino acid changes are obtained by affinity maturation. An “affinity matured” polypeptide is one having one or more alteration(s) in one or more residues which results in an improvement in the affinity of the orthogonal polypeptide for the cognate orthogonal receptor, or vice versa, compared to a parent polypeptide which does not possess those alteration(s). Affinity maturation can be done to increase the binding affinity by at least about 10% to 50-100-150% or more, or from 1 to 5 fold as compared to the “parent” polypeptide. An engineered orthogonal cytokine of the present invention activates the orthogonal receptor, as discussed above, but has significantly reduced binding and activation of the native receptor, for example an orthogonal cytokine may exhibit less than about 5% inhibition in a competitive inhibition with the corresponding native cytokine when assessed by ELISA and/or FACS analysis using sufficient amounts of the molecules under suitable assay conditions.

In some embodiments of the invention, the orthogonal receptor is a chain of the IL-2 receptor, i.e. a polypeptide selected from interleukin 2 receptor alpha (IL-2Rx; CD25), interleukin 2 receptor beta (IL-2Rβ; CD122), and interleukin 2 receptor gamma (IL-2Ry; CD132; common gamma chain). In some specific embodiments the orthogonal receptor is CD132, which is involved in signaling from IL-2, IL-4, IL-7 and IL-15. In other specific embodiments, the orthogonal receptor is CD122, which is involved in signaling from IL-2 and IL-15. The orthogonal receptor is usually paired with a counterpart orthogonal cytokine, e.g. IL-2, IL-4, IL-7, IL-15, etc.

In some specific embodiments, the orthogonal receptor is CD122. In some such embodiments, the orthogonal receptor is introduced into a T cell or NK cell that may also express CD25 and/or CD132. Nucleic acid coding sequences and protein compositions of the modified CD122 protein are provided. In the present invention CD122 is engineered to disrupt binding of the native cytokine by substituting an amino acid of the native sequence with a non-native amino acid, or by deletion of a native amino acid, at a position involved in binding to native IL-2. In some embodiments, the amino acid is substituted with a non-conservative change. Positions of interest for substitution or deletion include, without limitation, in human CD122 (hCD122) R41, R42, Q70, K71, T73, T74, V75, S132, H133, Y134, F135, E136, Q214. Positions of interest for substitution or deletion include, without limitation, in mouse CD122 (mCD122) R42, F67, Q71, S72, T74, S75, V76, S133, H134, Y135, I136, E137, R215.

In some embodiments, CD122 is substituted at one or a combination of positions selected from Q71, T74, H134, Y135 in the mouse protein; or Q70, T73, H133, Y134 in the human protein. In some embodiments, the engineered protein comprises amino acid substitutions at mCD122 H134 and Y135; or hCD122 H133 and Y134. In some embodiments the amino acid substitution is to an acidic amino acid, e.g. aspartic acid and/or glutamic acid. Specific amino acid substitutions include, without limitation, mCD122 substitutions Q71Y; T74D; T74Y; H134D, H134E; H134K; Y135F; Y135E; Y135R; and hCD122 changes Q70Y; T73D; T73Y; H133D, H133E; H133K; Y134F; Y134E; Y134R. The selection of an orthogonal cytokine may vary with the choice of orthogonal receptor.

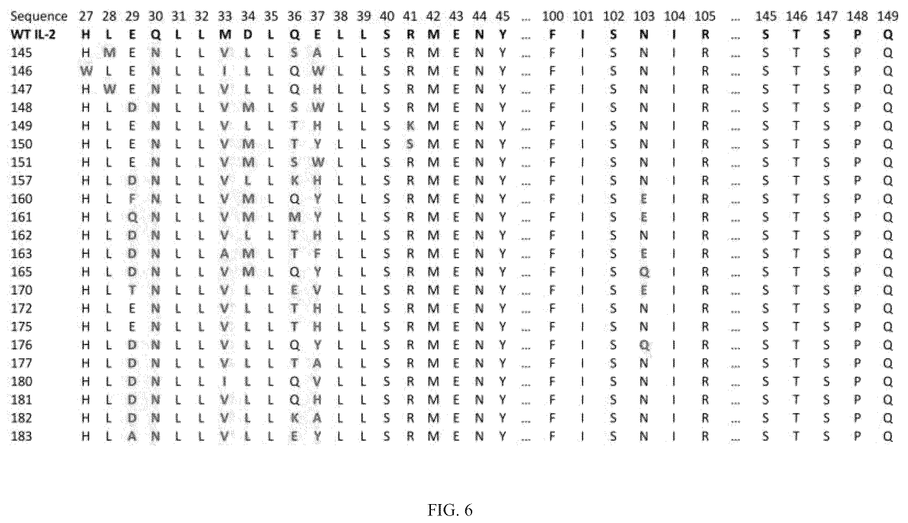

In some embodiments, where the orthogonal receptor is CD122, the orthogonal cytokine is IL-2, or IL-15. The cytokine can be selected for binding to the orthogonal receptor, e.g. by yeast display evolution, error-prone or targeted mutagenesis, and the like. A representative set of selected orthogonal sequences is shown in FIG. 6.

In some embodiments, the orthogonal cytokine is IL-2. In some embodiments, one or more of the following amino acid residues are substituted with an amino acid other than that of the native protein, or are deleted at that position: for mouse IL-2 (mIL-2) any one of H27, L28, E29, Q30, M33, D34, Q36, E37, R41, N103; for human IL-2 (hIL-2) any one of Q13, L14, E15, H16, L19, D20, Q22, M23, G27, R81, N88. In some such embodiments, the set of amino acid substitutions are selected from one or more of (for mIL-2) E29, Q30, M33, D34, Q36, and E37; and for hIL-2, E15, H16, L19, D20, Q22, M23, R81.

In some embodiments, the amino acid substitution for mIL-2 is one or more of: [H27W], [L28M, L28W], [E29D, E29T, E29A], [Q30N], [M33V, M331, M33A], [D34L, D34M], [Q36S, Q36T, Q36E, Q36K, Q36E], [E37A, E37W, E37H, E37Y, E37F, E37A, E37Y], [R41K, R41S], [N103E, N103Q]; and for hIL-2 is one or more of: [Q13W], [L14M, L14W], [E15D, E15T, E15A, E15S], [H16N, H16Q], [L19V, L19I, L19A], [D20L, D20M], [Q22S, Q22T, Q22E, Q22K, Q22E], [M23A, M23W, M23H, M23Y, M23F, M23Q, M23Y], [G27K, G27S], [R81D, R81Y], [N88E, N88Q], [T511]. In some embodiments the set of amino acid substitutions comprises one of the following sets of substitutions for mIL-2: [Q30N, M33V, D34N, Q36T, E37H, R41K]; [E29D, Q30N, M33V, D34L, Q36T, E37H]; [E29D, Q30N, M33V, D34L, Q36T, E37A], and [E29D, Q30N, M33V, D34L, Q36K, E37A] and for hIL-2: [H16N, L19V, D20N, Q22T, M23H, G27K]; [E15D, H16N, L19V, D20L, Q22T, M23H]; [E15D, H16N, L19V, D20L, Q22T, M23A], and [E15D, H16N, L19V, D20L, Q22K, M23A]; or a conservative variant thereof.

In some embodiments the amino acid substitution for hIL-2 is one or more of: [E15S, E15T, E15Q, E15H]; [H16Q]; [L19V, L19I]; [D20T, D20S, D20M, D20L]; [Q22K, Q22N]; [M23L, M23S, M23V, M23T]. In some embodiments a consensus set of mutations for hIL-2 is [E15S, H16Q, L19V, D20T/S/M; Q22K; M23L/S]. In some embodiments a consensus set of mutations for hIL-2 is [E15S, H16Q, L19V, D20L, M23 Q/A] and optionally Q22K.

In some embodiments the set of amino acid substitutions comprises one of the following sets of substitutions for hIL-2: [E15S; H16Q; L19V, D20T/S; Q22K, M23L/S]; [E15S; H16Q; L19I; D20S; Q22K; M23L]; [E15S; L19V; D20M; Q22K; M23S]; [E15T; H16Q; L19V; D20S; M23S]; [E15Q; L19V; D20M; Q22K; M23S]; [E15Q; H16Q; L19V; D20T; Q22K; M23V]; [E15H; H16Q; L19I; D20S; Q22K; M23L]; [E15H; H16Q; L19I; D20L; Q22K; M23T]; [L19V; D20M; Q22N; M23S]; [E15S, H16Q, L19V, D20L, M23Q, R81D, T51], [E15S, H16Q, L19V, D20L, M23Q, R81Y], [E15S, H16Q, L19V, D20L, Q22K, M23A], [E15S, H16Q, L19V, D20L, M23A].

Methods are provided for enhancing cellular responses, by engineering cells from a recipient or donor by introduction of an orthogonal receptor of the invention, and stimulating the orthogonal receptor by contacting the engineered cell with the cognate orthogonal cytokine. The subject methods include a step of obtaining the targeted cells, e.g. T cells, hematopoietic stem cells, etc., which may be isolated from a biological sample, or may be derived in vitro from a source of progenitor cells. The cells are transduced or transfected with an expression vector comprising a sequence encoding the orthogonal receptor, which step may be performed in any suitable culture medium.

In some embodiments, an engineered cell is provided, in which the cell has been modified by introduction of an orthogonal receptor of the invention. Any cell can be used for this purpose. In some embodiments the cell is a T cell, including without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. TR1, natural T_{Reg}, inducible T_{Reg}; memory T cells, e.g. central memory T cells, effector memory T cells, NKT cells, γδ T cells; etc. In other embodiments the engineered cell is a stem cell, e.g. a hematopoietic stem cell, or an NK cell. In some embodiments the cell is genetically modified in an ex vivo procedure, prior to transfer into a subject. The engineered cell can be provided in a unit dose for therapy, and can be allogeneic, autologous, etc. with respect to an intended recipient.

Cells, e.g. cells collected from a subject, may be separated from a mixture of cells by techniques that enrich for desired cells. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. Alternatively an engineered cell line, expanded allogeneic cells, and the like, are used for engineering.

Techniques for affinity separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

The collected and optionally enriched cell population may be used immediately, or may be frozen at liquid nitrogen temperatures and stored, being thawed and capable of being reused. The cells will usually be stored in 10% DMSO, 50% FCS, 40% RPMI 1640 medium.

In some embodiments a vector comprising a coding sequence that encodes the orthogonal receptor is provided, where the coding sequence is operably linked to a promoter active in the desired cell. Various vectors are known in the art and can be used for this purpose, e.g. viral vectors, plasmid vectors, minicircle vectors, which vectors can be integrated into the target cell genome, or can be episomally maintained. The receptor encoding vector may be provided in a kit, combined with a vector encoding an orthogonal cytokine that binds to and activates the receptor. In some embodiments the coding sequence for the orthogonal cytokine is operably linked to a high expression promoter, and may be optimized for production. In other embodiments, a kit is provided in which the vector encoding the orthogonal receptor is provided with a purified composition of the orthogonal cytokine, e.g. in a unit dose, packaged for administration to a patient.

In some embodiments a therapeutic method is provided, the method comprising introducing into a recipient in need thereof of an engineered cell population, wherein the cell population has been modified by introduction of a sequence encoding an orthogonal receptor of the invention. The cell population may be engineered ex vivo, and is usually autologous or allogeneic with respect to the recipient. In some embodiments, the introduced cell population is contacted with the cognate orthogonal cytokine in vivo, following administration of the engineered cells. An advantage of the present invention is a lack of cross-reactivity between the orthogonal cytokine and the native receptor.

Where the cells are contacted with the orthogonal cytokine in vitro, the cytokine is added to the engineered cells in a dose and for a period of time sufficient to activate signaling from the receptor, which may utilize the native cellular machinery, e.g. accessory proteins, co-receptors, and the like. Any suitable culture medium may be used. The cells thus activated may be used for any desired purpose, including experimental purposes relating to determination of antigen specificity, cytokine profiling, and the like, and for delivery in vivo.

Where the contacting is performed in vivo, an effective dose of engineered cells, including without limitation CAR-T cells modified to express an orthogonal IL-2β receptor, are infused to the recipient, in combination with or prior to administration of the orthogonal cytokine, e.g. IL-2 and allowed to contact T cells in their native environment, e.g. in lymph nodes, etc. Dosage and frequency may vary depending on the agent; mode of administration; nature of the cytokine; and the like. It will be understood by one of skill in the art that such guidelines will be adjusted for the individual circumstances. The dosage may also be varied for localized administration, e.g. intranasal, inhalation, etc., or for systemic administration, e.g. i.m., i.p., i.v., and the like. Generally at least about 10⁴ engineered cells/kg are administered, at least about 10⁵ engineered cells/kg; at least about 10⁶ engineered cells/kg, at least about 10⁷ engineered cells/kg, or more.

Where the engineered cells are T cells, an enhanced immune response may be manifest as an increase in the cytolytic response of T cells towards the target cells present in the recipient, e.g. towards elimination of tumor cells, infected cells; decrease in symptoms of autoimmune disease; and the like.

Engineered T cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow infusion), intraarterial, intraperitoneal, intrathecal or subcutaneous administration.

The engineered T cells may be infused to the subject in any physiologically acceptable medium, normally intravascularly, although they may also be introduced into any other convenient site, where the cells may find an appropriate site for growth. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection.

For example, typical ranges for the administration of cells for use in the practice of the present invention range from about 1×10⁵ to 5×10⁸ viable cells per kg of subject body weight per course of therapy. Consequently, adjusted for body weight, typical ranges for the administration of viable cells in human subjects ranges from approximately 1×10⁶ to approximately 1×10¹³ viable cells, alternatively from approximately 5×10⁶ to approximately 5×10¹² viable cells, alternatively from approximately 1×10⁷ to approximately 1×10¹² viable cells, alternatively from approximately 5×10⁷ to approximately 1×10¹² viable cells, alternatively from approximately 1×10⁸ to approximately 1×10¹² viable cells, alternatively from approximately 5×10⁸ to approximately 1×10¹² viable cells, alternatively from approximately 1×10⁹ to approximately 1×10¹² viable cells per course of therapy. In one embodiment, the dose of the cells is in the range of 2.5-5×10⁹ viable cells per course of therapy.

A course of therapy may be a single dose or in multiple doses over a period of time. In some embodiments, the cells are administered in a single dose. In some embodiments, the cells are administered in two or more split doses administered over a period of 1, 2, 3, 4, 5, 6, 7, 8, 9, 10, 11, 12, 13, 14, 21, 28, 30, 60, 90, 120 or 180 days. The quantity of engineered cells administered in such split dosing protocols may be the same in each administration or may be provided at different levels. Multi-day dosing protocols over time periods may be provided by the skilled artisan (e.g. physician) monitoring the administration of the cells taking into account the response of the subject to the treatment including adverse effects of the treatment and their modulation as discussed above.

For example, in the current clinical practice of CAR-T cell therapy, CAR-T cells are commonly administered in combination with lymphodepletion (e.g. by administration of Alemtuzumab (monoclonal anti-CD52), purine analogs, and the like) to facilitate expansion of the CAR-T cells to prior to host immune recovery. In some embodiments, the CAR-T cells may be modified for resistance to Alemtuzumab. In one aspect of the invention, the lymphodepletion currently employed in association with CAR-T therapy may be obviated or reduced by the orthogonal ligand expressing CAR-Ts of the present invention. As noted above, the lymphodepletion is commonly employed to enable expansion of the CAR-T cells. However, the lymphodepletion is also associated with major side effects of CAR-T cell therapy. Because the orthogonal ligand provides a means to selectively expand a particular T-cell population, the need for lymphodepletion prior to administration of the orthogonal ligand expressing CAR-Ts may be reduced. The present invention enables the practice of CAR-T cell therapy without or with reduced lymphodepletion prior to administration of the orthogonal ligand expressing CAR-Ts.

In one embodiment, the present invention provides a method of treating a subject suffering from a disease, disorder or condition amendable to treatment with CAR-T cell therapy (e.g. cancer) by the administration of a orthogonal ligand expressing CAR-Ts in the absence of lymphodepletion prior to administration of the orthogonal ligand CAR-Ts. In one embodiment, the present invention provides for a method of treatment of a mammalian subject suffering from a disease, disorder associated with the presence of an aberrant population of cells (e.g. a tumor) said population of cells characterized by the expression of one or more surface antigens (e.g. tumor antigen(s)), the method comprising the steps of (a) obtaining a biological sample comprising T-cells from the individual; (b) enriching the biological sample for the presence of T-cells; (c) transfecting the T-cells with one or more expression vectors comprising a nucleic acid sequence encoding a CAR and a nucleic acid sequence encoding an orthogonal receptor, the antigen targeting domain of the CAR being capable of binding to at least one antigen present on the aberrant population of cells; (d) expanding the population of the orthogonal receptor expressing CAR-T cells ex vivo; (e) administering a pharmaceutically effective amount of the orthogonal receptor expressing CAR-T cells to the mammal; and (f) modulating the growth of the orthogonal receptor expressing CAR-T cells using an ligand that binds selectively to the orthogonal receptor expressed on the CAR-T cell. In one embodiment, the foregoing method is associated with lymphodepletion or immunosuppression of the mammal prior to the initiation of the course of CAR-T cell therapy. In another embodiment, the foregoing method is practiced in the absence of lymphodepletion and/or immunosuppression of the mammal.

The preferred formulation depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Formulations to be used for in vivo administration are typically sterile. Sterilization of the compositions of the present invention may readily accomplished by filtration through sterile filtration membranes.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249:1527, 1990 and Hanes, Advanced Drug Delivery Reviews 28:97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Also provided are kits for use in the methods. The subject kits include an expression vector encoding an orthogonal cytokine receptor, or a cell comprising the expression vector. Kits may further comprise the cognate orthogonal cytokine. In some embodiments, the components are provided in a dosage form (e.g., a therapeutically effective dosage form), in liquid or solid form in any convenient packaging (e.g., stick pack, dose pack, etc.). Reagents for the selection or in vitro derivation of cells may also be provided, e.g. growth factors, differentiation agents, tissue culture reagents; and the like.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

In some embodiments the subject compositions, methods and kits are used to enhance a T cell mediated immune response. In some embodiments the immune response is directed towards a condition where it is desirable to deplete or regulate target cells, e.g., cancer cells, infected cells, immune cells involved in autoimmune disease, etc.

In some embodiments the condition is a chronic infection, i.e. an infection that is not cleared by the host immune system within a period of up to 1 week, 2 weeks, etc. In some cases, chronic infections involve integration of pathogen genetic elements into the host genome, e.g. retroviruses, lentiviruses, Hepatitis B virus, etc. In other cases, chronic infections, for example certain intracellular bacteria or protozoan pathogens, result from a pathogen cell residing within a host cell. Additionally, in some embodiments, the infection is in a latent stage, as with herpes viruses or human papilloma viruses.

The methods of the invention provide for a more effective killing of infected cells by the T effector cells of the host organism, relative to removal in the absence of treatment, and thus may be directed to the intracellular phase of the pathogen life cycle. The methods may further include monitoring the patient for efficacy of treatment. Monitoring may measure clinical indicia of infection, e.g. fever, white blood cell count, etc., and/or direct monitoring for presence of the pathogen.

In some embodiments the condition is cancer. The term “cancer”, as used herein, refers to a variety of conditions caused by the abnormal, uncontrolled growth of cells. Cells capable of causing cancer, referred to as “cancer cells”, possess characteristic properties such as uncontrolled proliferation, immortality, metastatic potential, rapid growth and proliferation rate, and/or certain typical morphological features. A cancer can be detected in any of a number of ways, including, but not limited to, detecting the presence of a tumor or tumors (e.g., by clinical or radiological means), examining cells within a tumor or from another biological sample (e.g., from a tissue biopsy), measuring blood markers indicative of cancer, and detecting a genotype indicative of a cancer. However, a negative result in one or more of the above detection methods does not necessarily indicate the absence of cancer, e.g., a patient who has exhibited a complete response to a cancer treatment may still have a cancer, as evidenced by a subsequent relapse.

The invention now being fully described, it will be apparent to one of ordinary skill in the art that various changes and modifications can be made without departing from the spirit or scope of the invention.

### EXPERIMENTAL

### Orthogonal IL-2 and IL-2Rβ

Here, we describe an invention involving engineered cytokines and receptors that enables selective expansion of desired cell subsets in settings of ex vivo adoptive cell therapy. The specific invention is described for the cytokine interleukin-2 (IL-2) and its receptor the IL-2R B chain (IL-2Rβ), which enables the specific expansion of T cells in adoptive cell therapy, and thus addresses an unmet need in immunotherapy. The approach described herein can be generalized to any setting of adoptive cell therapy where cells are stimulated by a specific receptor-ligand pair, including bone marrow and stem cell transplantation, and many other modalities.

Specifically described are orthogonal IL-2 and IL-2Rβ ligand-receptor pairs. Ortholog versions of IL-2 and ortholog versions of the IL-2Rβ bind specifically to each other, but not their wild-type counterpart. Multiple orthogonal IL-2 variant sequences are provided, with various degrees of affinity for orthogonal IL-2Rβ. Orthogonal IL-2 dependent signaling and T cell proliferation of T cells engineered to express orthogonal IL-2Rβ is shown.

IL-2 is an attractive biologic for the treatment of cancer and autoimmunity due to its ability to promote the expansion of effector T cells and regulatory T cells, respectively. However, this pleiotropic nature of IL-2 as well as off target toxicities limit its use in the clinic. The ability to decouple the immunostimulatory and immunoinhibitory properties of IL-2 can provide a superior form of IL-2 immunotherapy.

Demonstrated herein is the ability to engineer T cells to express the orthogonal IL-2Rβ. These engineered T cells are shown to respond to orthogonal IL-2, resulting in phosphorylation of downstream signal transduction molecules (e.g. STAT5), and T cell proliferation. The activity of orthogonal IL-2 on wild-type T cells is either completely abrogated or significantly blunted compared to the activity wild-type IL-2. Thus, selective T cell expansion using orthogonal IL-2/IL-2 receptor pairs is demonstrated.

Applications of orthogonal IL-2/IL-2 receptor pairs include but are not limited to the selective expansion of tumor reactive cytotoxic T cells for cancer therapy, NK cells for infectious disease and/or cancer, and regulatory T cells for autoimmune disorders.

IL-2 variants with blunted affinity for the intermediate (IL-2Rβ and IL-2Rγ) or high-affinity wild-type IL-2 receptor (IL-2Rα, Rβ, Rγ) due to mutations that disrupt but do not fully ablate binding to IL-2Rβ are also useful for selectively targeting the activity of ortholog IL-2 towards IL-2Rα high cells, e.g. in the treatment of autoimmune disease. IL-2 variants with ablated affinity for the IL-2Rβ chain, but that retain binding to IL-2Rα and therefore act as a competitive antagonist with wild-type IL-2 by inhibiting high-affinity IL-2R formation are useful to treat autoimmunity or graft-v-host disease.

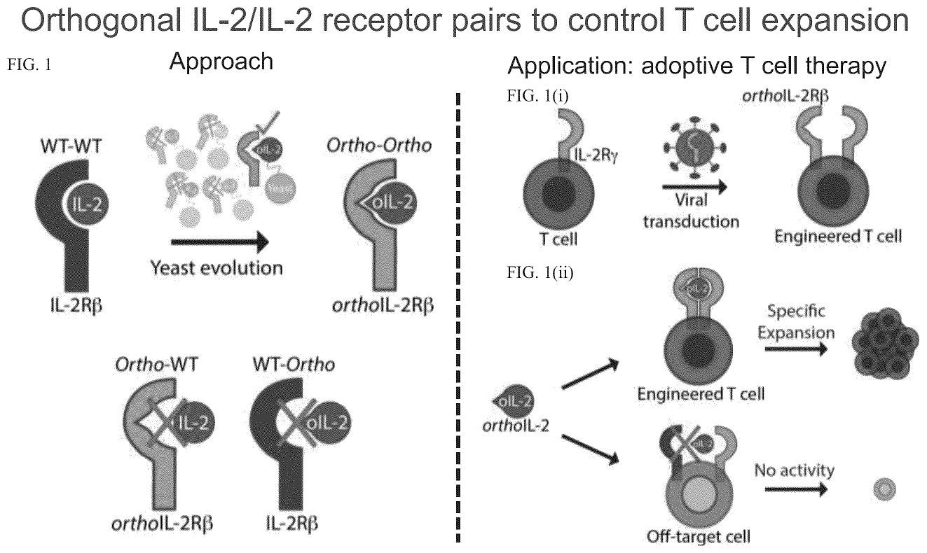

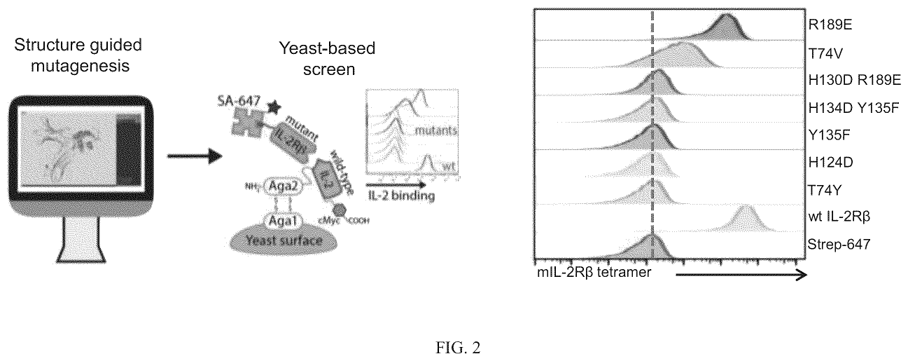

The overall concept of generating and utilizing orthogonal IL-2/IL-2 receptor pairs to control T cell expansion are shown in the schematic of FIG. 1. FIG. 2 provides a work flow, including the steps of generating IL-2Rβ orthologs that lack binding to wild-type IL-2, using structure guided mutagenesis. Mutations predicted to disrupt IL-2Rβ binding to wild-type IL-2 are confirmed experimentally using a yeast based screening assay, and are further verified using purified recombinant protein via surface plasmon resonance. Using this approach a number of IL-2Rβ point mutations that disrupt binding to wild-type IL-2 are described and each of these receptor variants may function as the orthogonal receptor. Single point mutations may also be combined with one, two, or more additional point mutations, to generate a larger library of IL-2Rβ orthologs.

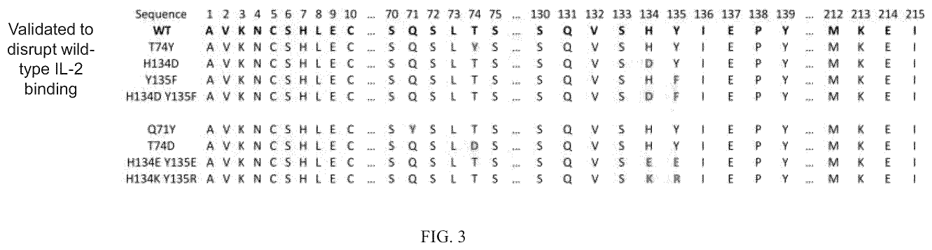

The sequences of orthogonal mouse IL-2Rβ variants are shown in FIG. 3. These mutations may be used as single point mutations, or any combination thereof, to generate IL-2Rβ orthologs with 1, 2, 3, or more point mutations, so long as the combined mutations disrupt wild-type IL-2 binding.

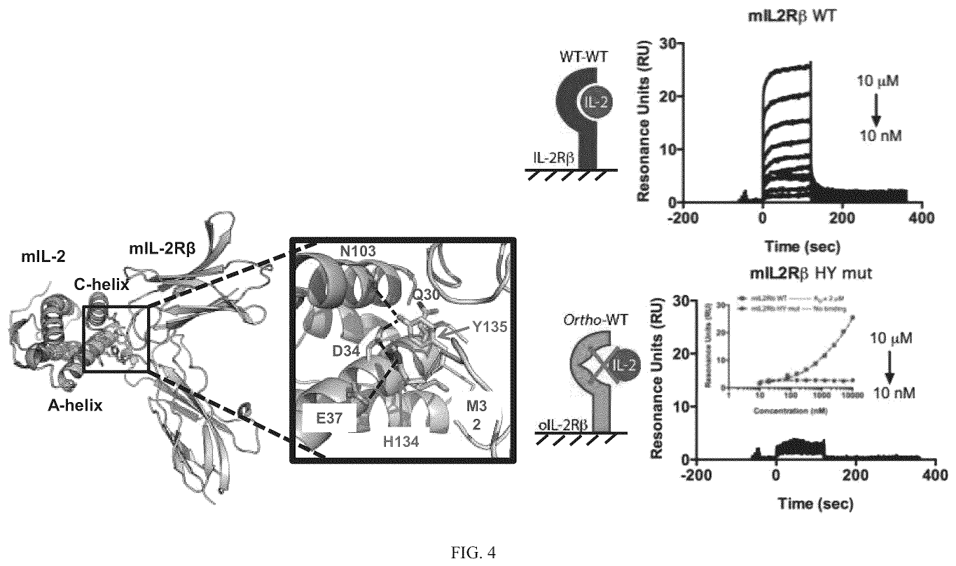

FIG. 4 shows the characterization of a mIL-2Rβ variant comprising the amino acid changes H134D, Y135F, which abrogate wild-type mIL-2 binding. These two residues are known IL-2 interaction hot spots (Ring A et al, Nat Immunol (2012) 13:1187-95) and we confirmed the mutations disrupt wild-type IL-2 binding via surface plasmon resonance (SPR).

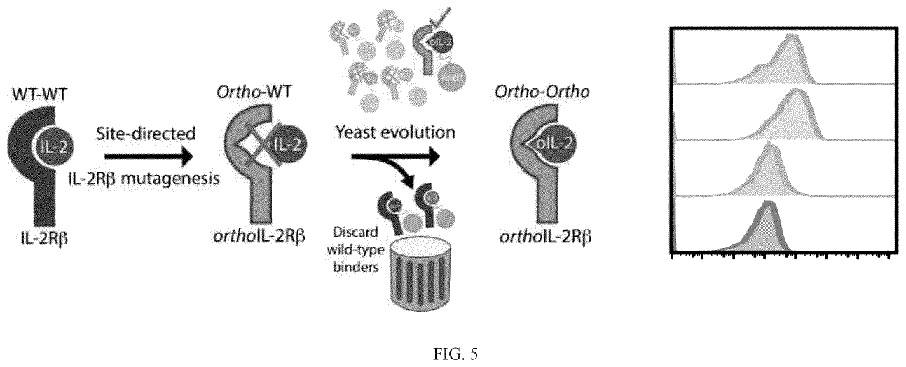

FIG. 5 illustrates the work flow for engineering orthogonal IL-2/IL-2Rβ pairs. An ortholog library is generated of IL-2 that randomizes residues in proximity to or in contact with the IL-2Rβ orthogonal ortholog amino acid residues. Using yeast display, select for IL-2 variants that bind ortholog IL-2Rβ, discard clones that bind wild-type IL-2Rβ. This process may be repeated using site-directed or error prone mutagenesis to generate IL-2 variants with differential binding properties to ortholog but not wild-type IL-2Rβ. Using this approach we generated a library of IL-2 orthologs that: 1) retain binding to the IL-2Rα chain (green curve) indicating intact structural integrity of the yeast-displayed orthogonal IL-2 variants, 2) bind to the orthogonal IL-2Rβ (orange curve) but, 3) not to the wild-type IL-2Rβ (blue curve).

Sequences of characterized orthogonal mouse IL-2 variants are shown in FIG. 6. An alignment of mouse IL-2 and IL-2Rβ and the human counterparts are shown in FIG. 15. These 4 sequences provide a reference for the native, or wild-type sequences. Amino acid residues that were altered to create orthogonal mouse IL-2/IL-2Rβ pairs are mainly conserved in humans. Therefore, the orthogonal mouse IL-2 and IL-2Rβ sequences can be readily translated to the human IL-2 and IL-2Rβ proteins.

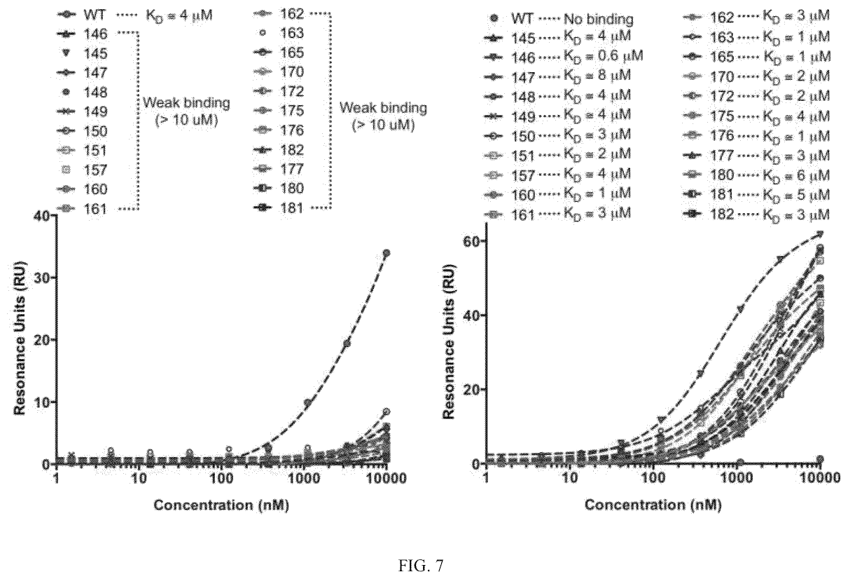

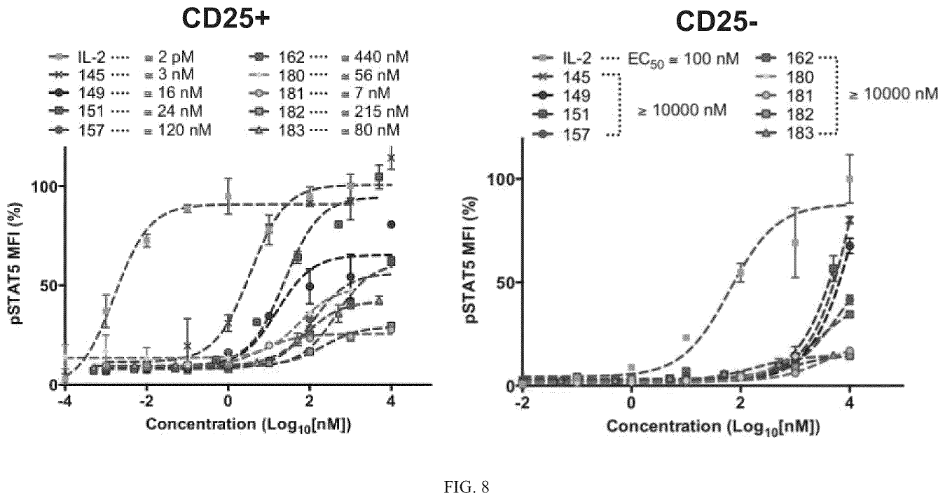

As shown in FIG. 7, orthoIL-2 variants bind orthoIL-2Rβ with affinity similar to or greater than the wild-type IL-2 and IL-2Rβ interaction. Soluble orthogonal IL-2 or wild-type IL-2 protein was flowed over a sensor chip coated with wild-type or orthoIL-2Rβ. Binding was determined by surface plasmon resonance (SPR) and curves were fitted using a 1:1 binding model. Shown in FIG. 8, orthoIL-2 variants exhibit blunted activity (phosphoSTAT5) on wild-type CD25 positive and CD25 negative splenocytes.

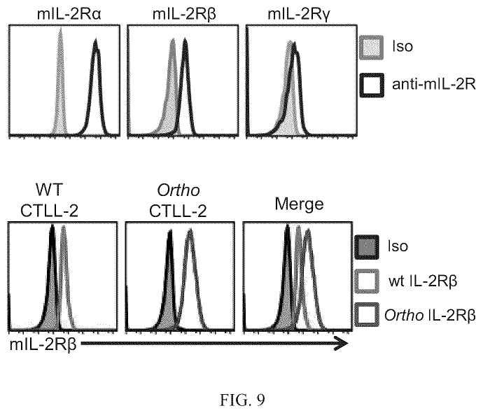

Generation of orthoIL-2Rβ expressing mouse CTLL-2 T cells is shown in FIG. 9. We created an immortalized mouse T cell line (CTLL-2) that expresses the orthogonal IL-2Rβ (orthoCTLL-2) via lentiviral transduction with of the gene encoding the full length orthogonal receptor. Transduced cells were selected with puromycin, which is toxin to untransduced cells, resulting in a stable CTLL-2 cell line that expresses both the wild-type and orthogonal IL-2Rβ. This cell line is also positive for CD25 and CD132 and thus represents T cells expressing the high-affinity IL-2 receptor complex. The antibody used to detect cell surface IL-2Rβ (CD122) does not discriminate between wild-type and orthoIL-2Rβ. Therefore, the increase in mean fluorescent intensity between wild-type and orthoIL-2Rβ CTLL-2 cells suggests that these cells express the orthogonal receptor. This is further supported by their resistance to puromycin, which is encoded by the same vector used to express the orthoIL-2Rβ.

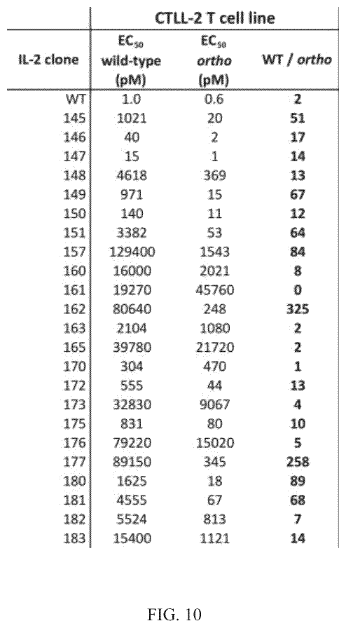

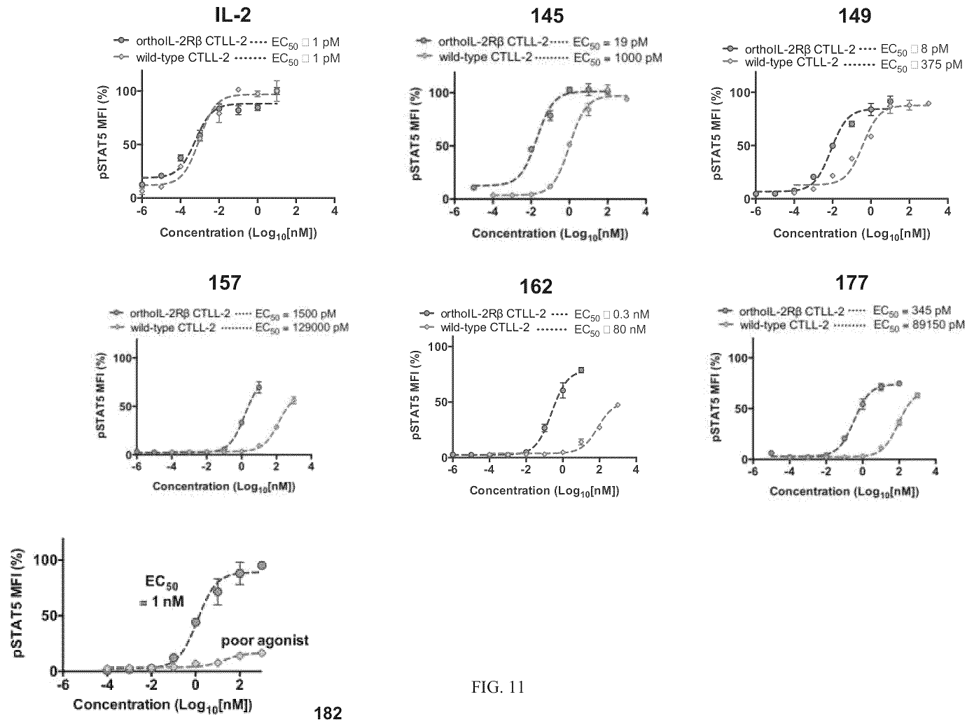

As shown in FIG. 10, the first set of orthoIL-2 variants are selective for ortho T cells. To interrogate orthogonal IL-2 signaling, we utilized our CTLL-2 cell model, either un-manipulated (wild-type) or transduced to also express the orthogonal IL-2Rβ (ortho). We then determined the ability of wild-type or various orthogonal IL-2 clones to induce phosphorylation of STAT5 (a quantitative readout of IL-2 dependent signal transduction). We identified a number of orthogonal IL-2 variants that induce selective STAT5 phosphorylation on orthogonal IL-2Rβ expressing cells compared to wild-type cells. Dose-response curves for select clones are shown on FIG. 11.

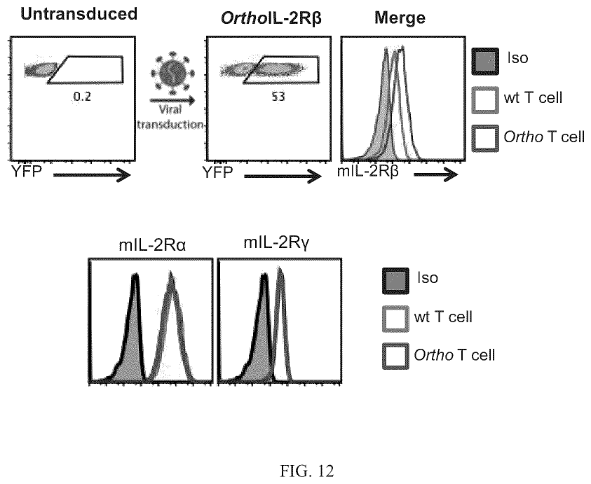

Primary lymph node derived T cells engineered to express orthoIL-2Rβ (H134D Y135F). In addition to our immortalized mouse T cell model, we have also generated orthoIL-2Rβ expressing primary mouse T cells by isolation of mouse lymph node and spleen cells, activation with CD3/CD28, followed by retroviral transduction of the gene encoding the full length orthogonal receptor. This construct also contains an IRES followed by the fluorescent protein YFP, which allows confirmation of transduction by analyzing YFP expression via FACS. The mouse T cells also express the high affinity IL-2 receptor complex (e.g. CD25, CD122, and CD132), shown in FIG. 12

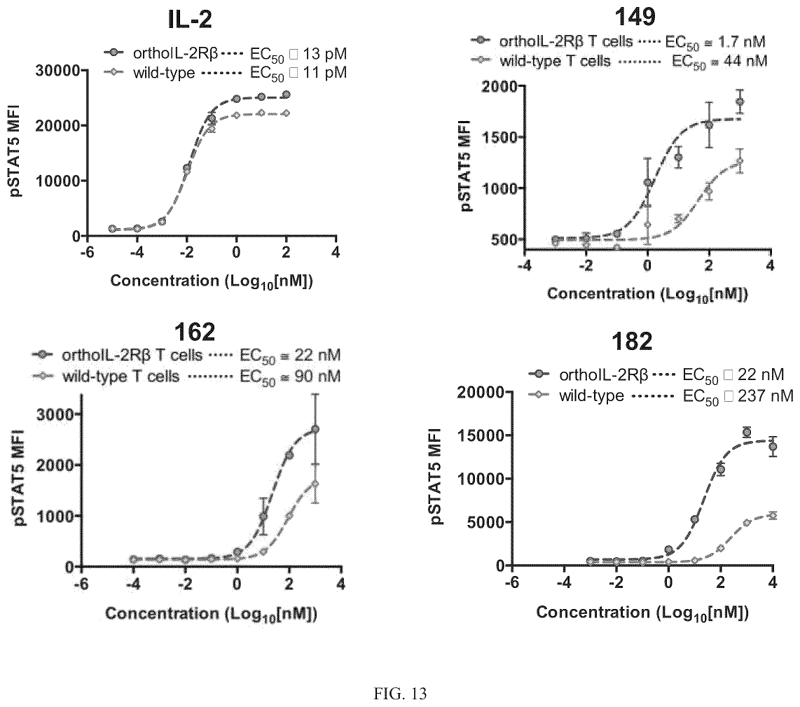

OrthoIL-2 variants induce selective STAT5 phosphorylation on orthoIL-2Rβ expressing primary mouse T cells, shown in FIG. 13.

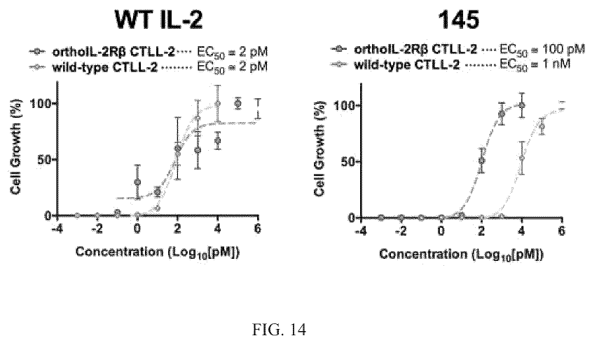

orthoIL-2 variants that selectively signal through the orthoIL-2Rβ (FIG. 11) also induce selective expansion of CTLL-2 cells expressing the orthoIL-2Rβ compared to wild-type CTLL-2 cells (FIG. 14).

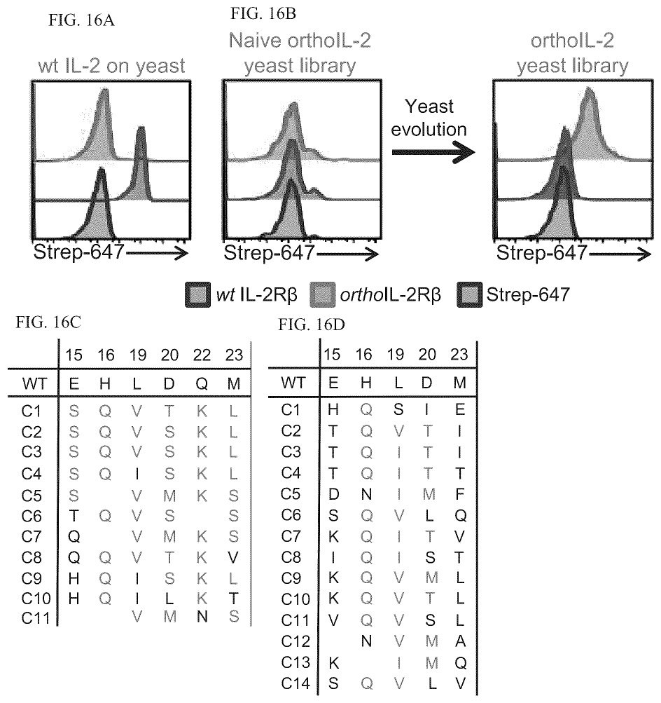

The orthogonal IL-2 engineering approach was also applied to human IL-2 and human IL-2Rβ. We introduced the H133D Y134F mutations that were used to create the mouse orthoIL-2Rβ into human IL-2Rβ, as these residues are highly conserved between mouse and human. Indeed, the wild-type hIL-2Rβ binds yeast-displayed wild-type IL-2, whereas the hIL-2Rβ H133D Y134F mutant (ortho-hIL-2Rβ) lacks detectable binding to wild-type IL-2 (FIG. 15). We created a library of human IL-2 mutants displayed on the surface of yeast by randomizing residues predicted to contact or be in proximity to the H133D Y134F mutations, and selected for IL-2 variants that bound the ortho but not wild-type human IL-2Rβ. This scheme was identical to what was employed to engineer mouse IL-2 orthogonal pairs, and was successful for the human pairs. The strategy is shown in FIG. 16. A consensus set of mutations were identified indicating a convergence of ortho hIL-2 sequences capable of binding the ortho hIL-2Rβ, shown in FIG. 16C.

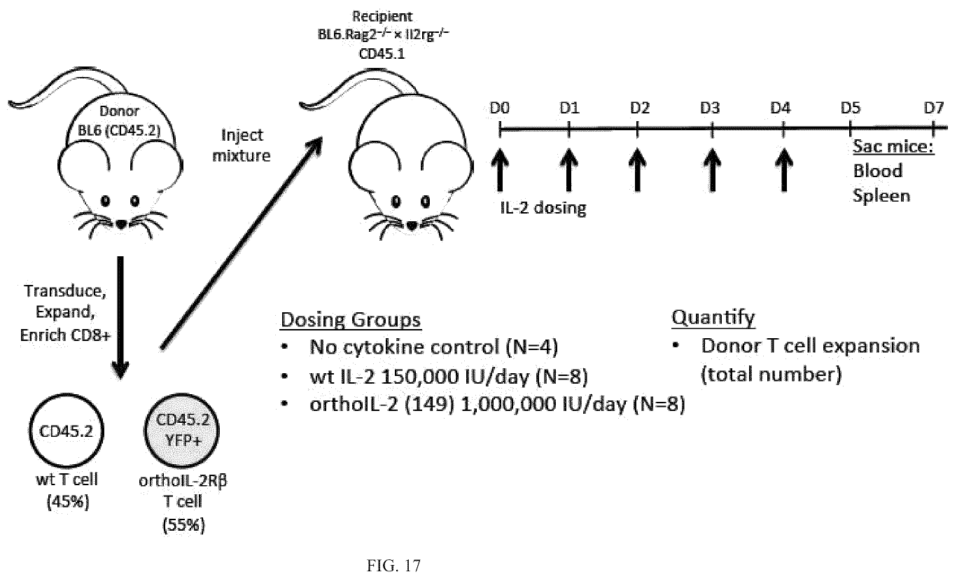

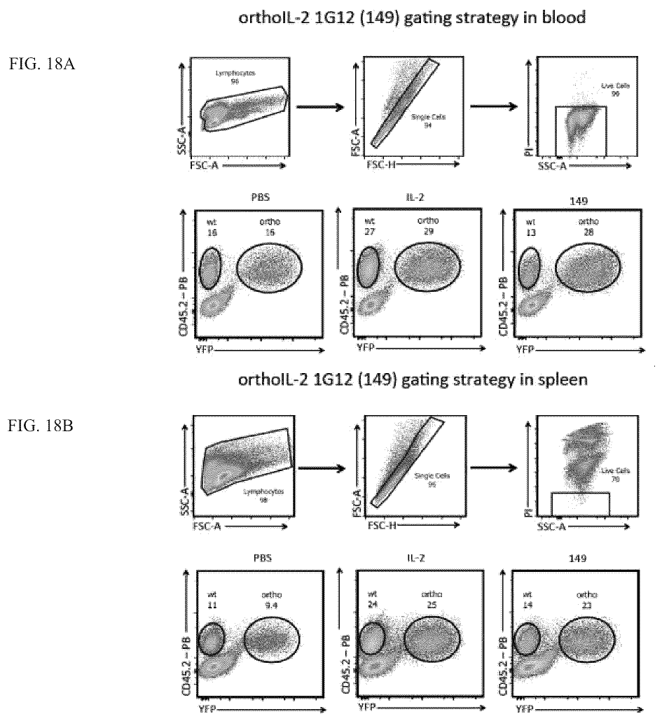

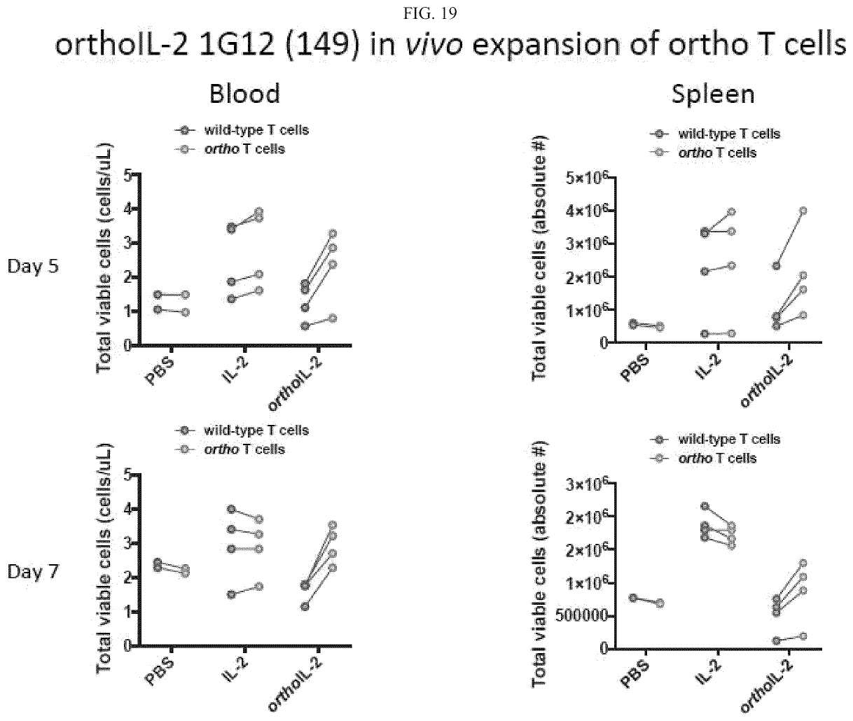

The polypeptides of the invention are also active in vivo. A mouse model was used to demonstrate selective expansion or increased survival of orthogonal IL-2Rb expressing T cells in mice, shown in FIGS. 17-19. It is shown that orthoIL-2 clone 1G12/149 selectively expands orthogonal but not wild-type T cells in mice. Treatment with wild-type IL-2 results in expansion of both wild-type and ortho T cells compared to a PBS control, whereas treatment with orthoIL-2 clone 1G12/149 selectively expands ortho T cells with limited activity on wild-type T cells.

### Example 2

### Human IL-2 Orthologs

### Materials and Methods

Protein production. DNA encoding wild-type human IL-2 was cloned into the insect expression vector pAcGP67-A, which includes a C-terminal 8xHIS tag for affinity purification. DNA encoding mouse serum albumin (MSA) was purchased from Integrated DNA Technologies (IDT, Coralville, Iowa 52241) and cloned into pAcGP67-A as a fusion between the N-terminus of hIL-2 and C-terminus of MSA. Variants of ortho human IL-2 isolated from the activity screen were synthesized as GBlocks (IDT) and cloned into the pAcGP67-A-MSA vector though overlap extension.

Insect expression DNA constructs were transfected into Trichoplusia ni (High Five®) cells (Invitrogen) using the BaculoGold® baculovirus expression system (BD Biosciences) for secretion and purified from the clarified supernatant via Ni-NTA followed by size exclusion chromatography with a Superdex-200 column and formulated in sterile Phosphate Buffer Saline (PBS). Proteins were concentrated and stored at −80° C.

Mammalian expression vectors. Full-length human CD25 was cloned into the lentiviral vector pCDH-CMV-MSC-EF1-Puro (System Biosciences). cDNA encoding full-length human IL-2Rβ was used as a template to clone the full length orthoIL-2Rβ by overlap extension PCR using mutagenic primers that introduce the H133D and Y134F mutations. The resulting PCR product was cloned into the retroviral vector pMSCV-MCS-IRES-YFP.

Cell culture. YT-NK-like cell line was generously provided by Dr. Junji Yodoi, Kyoto University. YT-cells were transduced with the pCDH-CMV-MSC-EF1-Puro-hCD25 lentivirus, and YT cells stably expressing full-length human CD25 (YT+) were selected in 10 μg/mL puromycin. YT+ cells were transduced with retrovirus containing pMSCV-MCS-IRES-YFP-ortho-human-R, and sorted by FACS to enrich the YFP+ (ortho) population to purity. HEK293T cells were generously provided by Dr. Irving Weissman's laboratory at Stanford University. HEK293T cells were maintained in DMEM supplemented with 10% Fetal Bovine Serum (FBS), 1% L-glutamine (L-glu), and 1% penicillin and streptomycin (P/S). YT cells were maintained in RPMI complete (RPMI+GlutaMax+10% FBS, 1% L-Glu, 1% NaPyr, 1% NEAA, 18 mM HEPES and 1% pen/strep).

Lentivirus and Retrovirus production. Lentivirus was produced in HEK293T cells using 3rd generation packaging vectors. Briefly, HEK293T cells were seeded at a density of 5×106 cells per 10 cm tissue culture dish and allowed to adhere for 5-7 hr in complete media (DMEM, 10% FBS, 1% L-Glu, 1% Pen/Strep). Supernatant was removed and replenished with low FBS (5%) DMEM (10 mL) and cells were transfected with a 4:2:1 ratio of pCDH:psPAX2:pMD2G using X-tremeGENER HP DNA transfection reagent (Sigma Aldrich) following the manufactures recommendation and cultured overnight at 37° C. in complete media. Media was removed and replenished with 7.5 mL of low FBS DMEM (DMEM, 5% FBS, 1% L-Glu, 1% P/S) and lentivirus was collected from the supernatant 24 and 48 hours later, pooled, clarified through a 0.45 μm filter, precipitated with PEG-it virus precipitation solution (System Bio), pelleted, re-suspended in complete media at 1/100th of the original volume, flash frozen in liquid nitrogen, and stored at −80° C.

Retrovirus was produced in HEK293T cells. Briefly, HEK293T cells were seeded at a density of 5×106 cells per 10 cm tissue culture dish and allowed to adhere for 5-7 hr in complete media (DMEM, 10% FBS, 1% L-Glu, 1% P/S). Supernatant was removed and replenished with low FBS DMEM (10 mL) and cells were transfected with a 1.5:1 ratio of pMSCV retroviral vector and pCL10A packaging vector (Novus Biologicals) (a kind gift of Dr. Melissa McCracken, Stanford University) using X-tremeGENER HP following the manufactures recommendation and cultured overnight in low FBS DMEM. Media was removed and replenished with 7.5 mL of low FBS DMEM and cultured for an additional 24 hr. Media was collected, clarified using a 0.45 μm filter, and flash frozen in liquid nitrogen for storage at −80 C. Media was replenished (low FBS DMEM) and cells were cultured for an additional 24 hr and virus was collected and stored as above.

Yeast display of IL-2. Human IL-2 was displayed on the surface of the yeast S. cerevisiae strain EBY100 by fusion to the C-terminus of Aga2 using the pCT302 vector harboring a 3C protease cleavage site between the C-terminus of Aga2 and the N-terminus of IL-2 as well as a N-terminal cMyc epitope tag. Briefly, competent EBY100 were electroporated with plasmid encoding yeast-displayed hIL-2 and recovered overnight in SDCAA selection media at 30° C. Transformed yeast were passaged once in SDCAA and yeast cultures in log phase were pelleted and resuspended at an OD600 of 1.0 in SGCAA induction media containing 10% SDCAA and cultured for 24 hr at 20° C. Surface expression of functional hIL-2 was confirmed by FACS by staining yeast with an AlexaFluor® 488-labeled anti-cMyc mAb (1:100 dilution; Cell Signaling) and AlexaFluor® 647-labeled streptavidin (SA) tetramers of wild-type hIL-2Rβ (500 nM SA).

### Human IL-2 Mutant Yeast Display Library Generation.

Site-directed libraries were created by assembly PCR using primers with the following degenerate codons: Library 3 (E15, H16, L19, D20, Q22, M23): (SEQ ID NO:10) 5′-CAAGTTCTACAAAGAAAACACAGCTACAACTGNHKNHKTTACTTNHKNHKTTANHKNHKATT TTGAATGGAATTAATAATTACAAGAATCCCAAACTC-3′ Library 4 (E15, H16, L19, D20, M23, N88): (SEQ ID NO:11) 5′-GTTCTACAAAGAAAACACAGCTACAACTGNHK NHKTTACTTNHKNHKTTACAGNHKATTTTGAATGGAATTAATAATTACAAGAATCC-3′, (SEQ ID NO: 12) 5′-CCCAGGGACTTAATCAGCNHKATCAACGTAATAGTTCTGGAACTAAAGGG-3′.

The following primers were used in all libraries: (SEQ ID NO:13) 5′-CGGTAGCGGTGGGGGGGGTTCTCTGGAAGTTCTGTTCCAGGGTCCGAGCGGCGGA-3′, (SEQ ID NO:14) 5′-GTAGCTGTGTTTTCTTTGTAGAACTTGAAGTAGGTGOGGATCCGC CGCTCGGACCCTGG-3′, (SEQ ID NO:15) 5′-CTTAAATGTGAGCATCCTGGTGAGTTT GGGATTCTTGTAATTATTAATTCCATTCAAAAT-3′, (SEQ ID NO:16) 5′-CCAGGATGCTCA CATTTAAGTTTTACATGCCCAAGAAGGCCACAG-3′, (SEQ ID NO:17) 5′-GAGGTTTGAGTT CTTCTTCTAGACACTGAAGATGTTTCAGTTCTGTGGCCTTCTTGGGC-3′, (SEQ ID NO:18) 5′-CAGTGTCTAGAAGAAGAACTCAAACCTCTGGAGGAAGTGCTAAATTTAGCTCAAAGC-3′, (SEQ ID NO:19) 5′-GATTAAGTCCCTGGGTCTTAAGTGAAAGTTTTTGCTTTGAGCTAAATT TAGCACTTCCTC-3′, (SEQ ID NO:20) 5′-CAGCATATTCACACATGAATGTTGTTTCAGATC CCTTTAGTTCCAGAACTATTACGTTG-3′, (SEQ ID NO:21) 5′-GAAACAACATTCATGTGTGAA TATGCTGATGAGACAGCAACCATTGTAGAATTTCTGAAC-3′, (SEQ ID NO:22) 5′-GAGATG ATGCTTTGACAAAAGGTAATCCATCTGTTCAGAAATTCTACAATGGTTGCTG-3′, (SEQ ID NO: 23) 5′-GATTACCTTTTGTCAAAGCATCATCTCAACACTAACTGCGGCCGCTTCTGGTGG CGAAC-3′, (SEQ ID NO:24) 5′-GATCTCGAGCAAGTCTTCTTCGGAGATAAGCTTTTGTTC GCCACCAGAAGCGG-3′.

The mutated IL-2 gene PCR product was assembled using Pfu Ultra DNA polymerase (Agilent) and an equal molar mixture of each primer. The product DNA was further PCR-amplified using the primers (SEQ ID NO:25) 5′-CGGTAGCGGTGGGGGGGGTTC-3′ and (SEQ ID NO:26) 5′-CGAAGAAGACTTGCTCGAGATC-3′ using Phusion DNA polymerase (NEB). The resulting assembled PCR product was gel purified and electroportated with linearized pCT302 vector into EBY-100 yeast to yield a library of ˜2×10⁸ transformants.

Evolution of orthogonal IL-2. Selection of yeast clones that specifically bind the orthoIL-2R were performed using a combination of magnetic activated cell sorting (MACS) and FACS. The first round of selection was performed with 2×10⁹ yeast, approximately 10 times the library diversity, to ensure 100% coverage of all transformants. The overall strategy employed was to first enrich the library for all full-length hIL-2 variants that bind the orthoIL-2Rβ (Round 1-3) with subsequent rounds using negative selection to remove IL-2 clones that bind wild-type IL-2Rβ and decreasing concentrations of orthoIL-2Rβ to enrich for IL-2 clones that bind the orthoIL-2Rβ with high affinity.

Yeast-based binding and functional screen. Single yeast clones were isolated via culture on SDCAA plates and single colony extraction or single cell FACS both into 96-well round-bottom tissue culture plates containing 100 μL SDCAA and cultured overnight at 30° C. in a shaking incubator. Yeast clones were expanded further in 1.5 mL SDCAA per well of a 96-deep well V-bottom plate for an additional 24 hours at 30° C. prior to induction in SGCAA media containing 10% SDCAA for 72 hr at 20° C. in a shaking incubator at a starting OD600 of 1.0, also in 1.5 mL and 96-deep well V-bottom plates. Induced yeast were pelleted, washed once with PBS, and resuspended in 200 μL/well of cleavage media (RPMI containing 25 mM HEPES, 0.2 mM TCEP, 20 μg/mL 3C protease) and incubated for 5 min at RT with agitation followed by an overnight incubation at 4° C. without agitation. Yeast were pelleted and the supernatant was clarified through a 96-well 0.45 μm cellulose acetate filter plates (Cat. 7700-2808, GE Heathcare). YT+ (wild-type and ortho expressing) and YT-were plated as described in the IL-2R signaling methods section and 50 μl of clarified yeast supernatant containing mutant IL-2 clones was added, incubated for 20 min at 37° C. and the reaction was terminated and pSTAT5 quantified as described below. The percentage of wild-type or ortho YT cells that are pSTAT5+ was quantified using FlowJo® (TreeStar Inc., Ashland OR) and used to select clones with selective or specific activity on ortho YT+ cells.

Retroviral transduction of human peripheral blood mononuclear cells (PBMCs). Leukoreduction chambers were acquired from the Stanford Blood Center. Blood was drained into a sterile 50 ml conical tube (˜7 ml) and PBS+2% FBS was added to 34 ml total. Density gradient medium (15 ml, Ficoll-Paque Plus, GE Healthcare, 17-1440-03) was loaded into two SepMate®-50 tubes (Stemcell, 15450) and 17 ml of diluted cells were gently pipetted on top. The SepMate® tubes were spun at 1200×g for 15 minutes at room temperature. The top layer, containing the PBMCs, was poured off into a new tube and RPMI was added to 50 ml. The cells were spun at 1200 rpm for 5 minutes to pellet. Pellets were resuspended in 10 ml ACK lysing buffer (Gibco A10492-01) for 4 minutes and quenched to 40 ml with RPMI complete. Cells were pelleted again, suspended in 15 ml of RPMI complete and counted. Cells (1×10⁶) were plated into each well of a 24 well tissue culture dish, and 25 μL of Dynabeads® Human T-Activator CD3/CD28 (Cat #11131D) and 100 U/ml hIL-2 was added to each well. Cells were allowed to activate at 37° C. in incubator for 48 hours.

Activated human PBMCs were transduced via spinfection (see Berggren W T, Lutz M, Modesto V. General Spinfection Protocol. 2012 Dec. 10. In: StemBook [Internet]. Cambridge (MA): Harvard Stem Cell Institute; 2008) using un-concentrated retroviral supernatant (˜ 2 mL per well) containing 10 μg/mL polybrene and 100 IU/mL hIL-2 for 1.5 hr at 32° C. and 2500 RPM. The viral supernatant was gently aspirated and replaced with fresh RPMI complete media containing 100 IU/mL hIL-2 and cultured for 24 hours at 37° C. Cells were harvested via gentle pipetting and Dynabeads® removed with a magnet. Cells were pelleted via centrifugation and re-suspended at a density of 1×10⁶ cells/mL in fresh RPMI complete media containing 100 IU/mL hIL-2 and expanded overnight at 37° C. prior to further downstream cellular assays.

IL-2R signaling via phosphorylation of STAT5. Quantification of IL-2 and orthoIL-2 signal transduction via intracellular pSTAT5 was done. Actively growing YT+ and YT+ ortho cells were pelleted, combined in a 50/50 ratio and plated at a density of 5×10⁵ cells per well of an ultra-low binding 96-well round bottom plate (Cat. 7007; Costar) in 50 μL warm media. Cells were stimulated by addition of 50 μL media containing serial dilutions of wild-type or ortho IL-2 for 20 min at 37° C., and the reaction was terminated by fixation with 1.5% paraformaldehyde for 10 min at room temperature (RT) with agitation. Cells were pelleted, decanted, and permeabilized with 200 μL of 100% ice-cold methanol for at least 30 min on ice or incubation at −80° C. overnight. Fixed, permeabilized cells were washed three times with FACS buffer and intracellular phosphorylated STAT5 was detected with AlexaFluor®647 labeled anti-STAT5 pY694 (BD Biosciences) diluted 1:50 in FACS buffer and incubated for 1 hour at 4 C in the dark. Cells were washed and analyzed on a CytoFLEX® equipped with a high-throughput autosampler (Beckman Coulter). Data represent the mean fluorescence intensity and points were fit to a log (agonist) vs. response (three parameters) model using Prism 5® (GraphPad). All data are presented as mean (n=3)±SD.

In vitro primary human PBMC proliferation assay. Human peripheral blood monocyte cells containing a mixture of wild-type and ortho transduced T cells were collected by centrifugation, re-suspended in RPMI complete media lacking hIL-2 and seeded at a density of 50,000 cells per/well (in 50 μL) in a 96-well round bottom tissue culture plate (day 1). Cell growth was stimulated by addition of serial dilutions of wild-type or orthoIL-2 (50 μL) to a total volume of 100 μL and cultured for 2 days at 37° C. On day 3, cells were fed fresh cytokine in an additional 100 UL volume and cultured for another 2 days. On day 5, 50 μL of DAPI was added to a final concentration of 0.5 μg/mL and cell counts for each test population were quantified by FACS using the CytoFLEX® equipped with a high throughput sampler. The total number of live cells in a set volume was derived after gating for live cells based on FSC and SSC and DAPI negative. Data were analyzed using FlowJo® (Tree Star Inc.). Data represent the total live cell count plotted vs. the concentration of cytokine, or as the ratio of ortho cells to total live cells plotted vs the concentration of cytokine. Data are presented as mean (n=4)+SD.

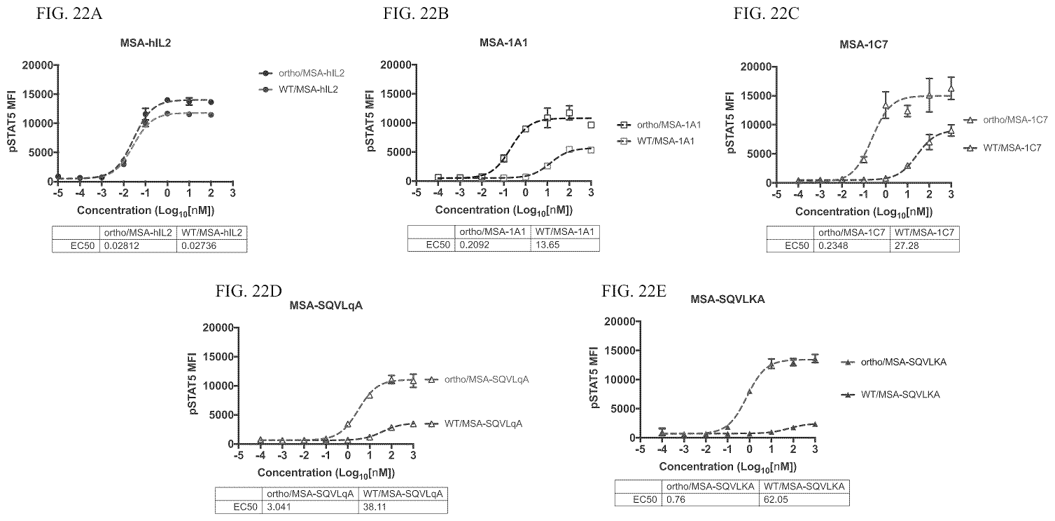

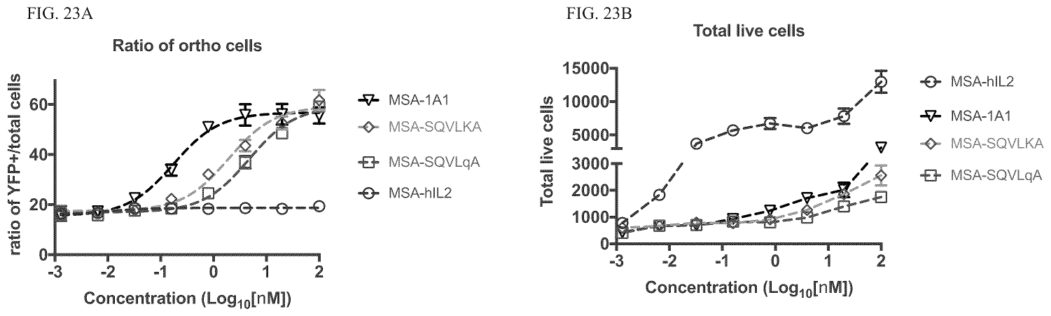

As shown in FIG. 22, ortho human IL-2 signals through the orthoIL-2R expressed in YT cells in vitro. As shown in FIG. 23, ortho human IL-2 preferentially expands human PBMCs expressing the ortho IL-2R. Human PBMCs were isolated, activated and transformed with retrovirus containing ortho human IL-2Rβ with an IRES YFP (YFP+). Initial ratio of YFP+ cells to total live cells was 20%. 5×10⁵ cells were plated with the indicated concentrations of MSA-human IL-2 (circles) or ortho variants MSA-SQVLKA (diamonds), MSA-SQVLqA (squares) or MSA-1A1 (black triangles) on day 1, and re-fed the same concentration on day 3. On day 5 the plate was read by flow cytometry. (A) The ratio of YFP+ (ortho expressing) cells to total live cells was calculated, and the mean (n=4)±SD was plotted versus the concentration (left). (B) Total live cell counts (mean (n=4)±SD) were also plotted versus the cytokine concentration (right). The orthogonal cytokines were did not support as much total cell growth as wild type MSA-hIL-2 at the same concentration, but were selective in strongly expanding the ortho-expressing T cells.

The amino acid substitutions made in the orthogonal hIL-2 proteins are shown below in Table 1.

TABLE 1

| hIL2 | E15 | H16 | L19 | D20 | Q22 | M23 | R81 | Other |
|---|---|---|---|---|---|---|---|---|
| 1A1 | S | Q | V | L | | Q | D | T51I |
| (MJH | | | | | | | | |
| lib 4) | | | | | | | | |
| 1C7 | S | Q | V | L | | Q | Y | |
| (MJH | | | | | | | | |
| lib 3) | | | | | | | | |
| SQVLKA | S | Q | V | L | K | A | | |
| (design) | | | | | | | | |
| SQVLqA | S | Q | V | L | | A | | |
| (design) | | | | | | | | |

What is claimed is:

1. An engineered mouse IL-2 polypeptide, modified at one or residues selected from H27, L28, E29, Q30, M33, D34, Q36, E37, R41, and N103.

2. A system for selective activation of a receptor in a cell, the system comprising:
(a) an orthogonal mouse CD122 receptor modified at one or more residues selected from R42, F67, Q71, S72, T74, S75, V76, S133, H134, Y135, I136, E137, R215; and
(b) the engineered mouse IL-2 polypeptide of claim 1.

3. The system of claim 2, wherein the orthogonal receptor is expressed by a mammalian cell.

4. The system of claim 3, wherein the cell is an immune cell or a stem cell.

5. The system of claim 4, wherein the immune cell is a T cell.

6. A pharmaceutical composition comprising the engineered mouse IL-2 polypeptide of claim 1; and a pharmaceutically acceptable excipient.

7. A kit comprising the system of claim 2.


SEQUENCE LISTING (26 sequences)

SEQ ID NO: 1 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 2 - protein, 513 aa, Mus musculus
AVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSHYIEPYLEFEARRRLLGHSWEDASVLSLKQRQQWLFLEMLIPSTSYEVQVRVKAQRNNTGTWSPWSQPLTFRTRPADPMKEILPMSWLRYLLLVLGCFSGFFSCVYILVKCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 3 - protein, 149 aa, Mus musculus
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 4 - protein, 133 aa, Homo sapiens [sequence omitted]

SEQ ID NO: 5 - protein, 133 aa, synthetic construct
APTSSSTKKTQLQLSQLLVLLQQILNGINNYKNPKLTRMLTFKFYMPKKAIELKHLQCLEEELKPLEEVLNLAQSKNFHLDPRDLISNINVIVLELKGSETTFMCEYADETATIVEFLNRWITFCQSIISTLT

SEQ ID NO: 6 - protein, 133 aa, synthetic construct
APTSSSTKKTQLQLSQLLVLLQQILNGINNYKNPKLTRMLTFKFYMPKKATELKHLQCLEEELKPLEEVLNLAQSKNFHLYPRDLISNINVIVLELKGSETTFMCEYADETATIVEFLNRWITFCQSIISTLT

SEQ ID NO: 7 - protein, 133 aa, synthetic construct
APTSSSTKKTQLQLSQLLVLLKAILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 133 aa, synthetic construct
APTSSSTKKTQLQLSQLLVLLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 9 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 10 - DNA, 98 bp, synthetic construct
  source 1..98: mol_type = other DNA
caagttctacaaagaaaacacagctacaactgnhknhkttacttnhknhkttanhknhkattttgaatggaattaataattacaagaatcccaaactc

SEQ ID NO: 11 - DNA, 88 bp, synthetic construct
  source 1..88: mol_type = other DNA
gttctacaaagaaaacacagctacaactgnhknhkttacttnhknhkttacagnhkattttgaatggaattaataattacaagaatcc

SEQ ID NO: 12 - DNA, 50 bp, synthetic construct
  source 1..50: mol_type = other DNA
cccagggacttaatcagcnhkatcaacgtaatagttctggaactaaaggg

SEQ ID NO: 13 - DNA, 55 bp, synthetic construct
  source 1..55: mol_type = other DNA
cggtagcggtgggggcggttctctggaagttctgttccagggtccgagcggcgga

SEQ ID NO: 14 - DNA, 59 bp, synthetic construct
  source 1..59: mol_type = other DNA
gtagctgtgttttctttgtagaacttgaagtaggtgcggatccgccgctcggaccctgg

SEQ ID NO: 15 - DNA, 60 bp, synthetic construct
  source 1..60: mol_type = other DNA
cttaaatgtgagcatcctggtgagtttgggattcttgtaattattaattccattcaaaat

SEQ ID NO: 16 - DNA, 45 bp, synthetic construct
  source 1..45: mol_type = other DNA
ccaggatgctcacatttaagttttacatgcccaagaaggccacag

SEQ ID NO: 17 - DNA, 59 bp, synthetic construct
  source 1..59: mol_type = other DNA
gaggtttgagttcttcttctagacactgaagatgtttcagttctgtggccttcttgggc

SEQ ID NO: 18 - DNA, 57 bp, synthetic construct
  source 1..57: mol_type = other DNA
cagtgtctagaagaagaactcaaacctctggaggaagtgctaaatttagctcaaagc

SEQ ID NO: 19 - DNA, 60 bp, synthetic construct
  source 1..60: mol_type = other DNA
gattaagtccctgggtcttaagtgaaagtttttgctttgagctaaatttagcacttcctc

SEQ ID NO: 20 - DNA, 59 bp, synthetic construct
  source 1..59: mol_type = other DNA
cagcatattcacacatgaatgttgtttcagatccctttagttccagaactattacgttg

SEQ ID NO: 21 - DNA, 60 bp, synthetic construct
  source 1..60: mol_type = other DNA
gaaacaacattcatgtgtgaatatgctgatgagacagcaaccattgtagaatttctgaac

SEQ ID NO: 22 - DNA, 58 bp, synthetic construct
  source 1..58: mol_type = other DNA
gagatgatgctttgacaaaaggtaatccatctgttcagaaattctacaatggttgctg

SEQ ID NO: 23 - DNA, 59 bp, synthetic construct
  source 1..59: mol_type = other DNA
gattaccttttgtcaaagcatcatctcaacactaactgcggccgcttctggtggcgaac

SEQ ID NO: 24 - DNA, 53 bp, synthetic construct
  source 1..53: mol_type = other DNA
gatctcgagcaagtcttcttcggagataagcttttgttcgccaccagaagcgg

SEQ ID NO: 25 - DNA, 21 bp, synthetic construct
  source 1..21: mol_type = other DNA
cggtagcggtgggggcggttc

SEQ ID NO: 26 - DNA, 22 bp, synthetic construct
  source 1..22: mol_type = other DNA
cgaagaagacttgctcgagatc